(19) United States
(12) Patent Application Publication
(10) Pub. No.: US 2026/0116946 A1
(43) Pub. Date: Apr. 30, 2026

(54) NOVEL CD200 FUSION PROTEINS

(71) Applicant: DUCENTIS BIOTHERAPEUTICS LIMITED, Bristol (GB)

(72) Inventor: Rebecca ASHFIELD, Bristol (GB)

(73) Assignee: DUCENTIS BIOTHERAPEUTICS LIMITED, Bristol (GB)

(21) Appl. No.: 18/863,179

(22) PCT Filed: May 5, 2023
(86) PCT No.: PCT/IB2023/054722

§ 371 Date: Nov. 5, 2024

(30) Foreign Application Priority Data
May 6, 2022 (GB) 2206673.2

(51) Int. Cl. C07K 14/705; A61K 31/52; A61K 31/573; A61K 38/13; A61P 29/00; A61P 37/08; C07K 16/28; C12N 15/62

(52) U.S. Cl. CPC C07K 14/70503; A61K 31/52; A61K 31/573; A61K 38/13; A61P 29/00; A61P 37/08; C07K 16/2809; C07K 16/2866; C12N 15/625; C07K 2319/02; C07K 2319/30

(57) ABSTRACT

The invention relates to fusion proteins comprising a mutated CD200 portion comprising K130Y and I131Y mutations which binds with greater affinity to the human CD200 receptor than wild-type CD200, directly fused to a non-CD200 IgG4 Fc fragment which comprises S228P, M428L and N434S mutations and deletion of the first 5 amino acids. The invention also relates to a polynucleotide encoding the fusion protein, a pharmaceutical composition comprising it and uses thereof.

### CROSS REFERENCE TO RELATED APPLICATION

This application is a 35 U.S.C. § 371 National Stage of International Patent Application No. PCT/IB2023/054722, filed May 5, 2023, claiming benefit from British Patent Application No. 2206673.2, filed May 6, 2022, the disclosures of which are incorporated herein in their entirety by reference, and priority is claimed to each of the foregoing.

### SEQUENCE LISTING STATEMENT

The instant application contains a Sequence Listing in electronic format which has been submitted via EFS-Web. Said Sequence Listing, created on Jun. 24, 2025, is named “4549-140.xml” and is 8, 192 bytes in size. The information in the electronic format of the Sequence Listing is part of the present application and is incorporated herein by reference in its entirety.

### FIELD OF THE INVENTION

The invention relates to fusion proteins comprising a mutated CD200 portion comprising K130Y and I131Y mutations which binds with greater affinity to the human CD200 receptor than wild-type CD200, directly fused to a non-CD200 IgG4 Fc fragment which comprises S228P, M428L and N434S mutations and deletion of the first 5 amino acids. The invention also relates to a polynucleotide encoding the fusion protein, a pharmaceutical composition comprising it and uses thereof.

### BACKGROUND OF THE INVENTION

Inflammatory diseases including autoimmunity and allergy are the second leading cause of chronic illness globally and in the U.S they are the leading cause of morbidity in women. According to a 2008 international survey, chronically ill patients in the US as compared with those in other countries are more likely to do without proper care due to the burden of cost (Schoen, C. et al., (2008) Health Affairs Web Exclusive, w1-w16). Additionally, these patients are more likely to experience the highest rates of medical errors, problems with coordination of care, and high out-of-pocket health care costs.

Currently, the American Autoimmune Related Disease Association (AARDA) estimates that 50 million Americans have an autoimmune disease. Epidemiological data are lacking to determine the full direct and indirect cost to the overall health care system due to autoimmune disease. However, in 2001, the National Institutes of Allergy and Infectious Diseases (NIAID) Director Dr Anthony Fauci estimated that annual autoimmune disease treatment costs were greater than $100 billion. While $100 billion is a staggering figure, it is likely a vast understatement of the true costs of autoimmune disease as the annual costs of only seven of the 100+ known autoimmune diseases, Crohn's disease, ulcerative colitis, systemic lupus erythematosus (SLE), multiple sclerosis (MS), rheumatoid arthritis (RA), psoriasis, and scleroderma, are estimated through epidemiological studies to total from $51.8-$70.6 billion annually. Furthermore, these estimates overlook the cost of immunosuppressive therapy during transplantation.

Autoimmune diseases are chronic conditions with no cure, which arise when the immune system decides that healthy cells are foreign and attacks them. Depending on the type, an autoimmune disease can affect one or many different types of body tissue and can cause abnormal organ growth and changes in organ function. The normal regulation of the immune system is largely due to receptor/ligand pairs that includes proteins that are expressed by cells involved in an immune response. However, these receptor/ligand pairs are often included in signalling cascades which contribute to the pathology of autoimmune disease.

OX-2 membrane glycoprotein, also named CD200 (Cluster of Differentiation 200), is a human protein encoded by the CD200 gene which is expressed in a variety of cell types (Barclay, A. N. (1981) Immunology 44, 727) and has a high degree of homology to molecules of the immunoglobulin gene family. The protein encoded by this gene is a type-1 membrane glycoprotein which contains two immunoglobulin domains and binds to the CD200 receptor (CD200R).

CD200R is expressed on myeloid cells (monocytes, macrophages, dendritic cells and eosinophils) and T cells (Wright, et al., (2000), Immunity 12, 233-242; Wright, et al., (2003), J. Immunol, 171, 3034-3046).

Engagement of CD200 with CD200R delivers an inhibitory signal to myeloid and T-cells, thus exerting an immunosuppressive effect on both the innate and adaptive arms of the immune system (Rahim S. A., (2005) AIDS, 19, 1907-1925; Shiratori, I., (2005) J. Immunol, 175, 4441-4449; Misstear, K., et al., (2012), Journal of Virology, 86 (11), 6246-6257).

CD200R agonists have been shown to reduce pathology in a wide range of murine disease models, for example arthritis (Gorczynski, et al., (2001) Clin. Immunol. 101, 328-34; Gorczynski, et al., (2002) Clin. Immunol. 104, 256-264), graft rejection (Gorczynski, et al., (2002) Transplantation 73, 1948-1953), failed pregnancy (Gorczynski, et al., (2002) Am. J. Reprod. Immunol., 48, 18-26), contact hypersensitivity (Rosenblum, et al., (2004) Blood 103, 2691-8), influenza induced lung inflammation (Snelgrove, et al., (2008) Nat. Immunol., 9, 1074-1083) and HSV-induced inflammatory lesions (Sarangi, et al., (2009) Clin. Immunol. 131, 31-40).

Additionally, CD200^{-/-} mice challenged with influenza virus developed more severe disease, which was associated with increased lung infiltration and lung endothelium damage, compared with wildtype controls (Rygiel. T. P., et al. (2009) J. Immunol. 183 (3), 1990-1996). CD200^{-/-} mice did develop immune responses that could control viral load, suggesting that the severe disease was caused by poor control of the immune response as opposed to the beneficial antiviral immune response. Disease could be prevented by T-cell depletion before viral challenge, despite the dramatically increased viral load that resulted. Rygiel. T. P., et al. (2009) concluded that T cells are essential for the manifestation of disease symptoms during influenza infection, and that lack of down-modulating CD200-CD200R signalling, rather than viral load, increases immune pathology.

Profiling studies have shown that hCD200 expression is down regulated in diverse patient populations, such as patients with multiple sclerosis (Koning, et al., (2007) Ann. Neurol. 62, 504-514), asthma exacerbation (Aoki, et al., (2009) Clin. Exp. Allergy 39, 213-221), Alzheimer's disease (Walker, et al., (2009) Exp. Neurol. 215, 5-19), primary hypertrophic osteoarthropathy (Ren, et al., (2013) Rheumatol. Int. 33 (10), 2509-2512), failed pregnancy (Clark (2009) Am. J. Reprod. Immunol. 61, 75-84) and lichen planopilaris (hair loss) (Harries, et al., (2013) J. Pathol. 231 (2), 236-247).

Agonist CD200 proteins are disclosed in, for example, WO 2000/061171 and WO 2008/089022. The literature describes the use of wild-type CD200 molecules to modulate immune cell function. The invention relates to mutant CD200 proteins which bind with greater affinity to the CD200 receptor than wild-type CD200.

Therapeutic intervention with molecules that modulate the CD200 pathway therefore offer a means of controlling exaggerated or unwanted immune responses and reducing pathology in patients suffering from chronic or intermittent (flare-up) autoimmune disease.

There is a need to provide improved clinical efficacy at lower doses and overcome the problems associated with currently available treatments to autoimmune diseases.

### SUMMARY OF THE INVENTION

According to a first aspect of the invention, there is provided a fusion protein comprising:
(i) a mutated CD200 portion comprising mutations at amino acid residue positions 130 and 131, wherein said mutations are K130Y and I131Y; and
(ii) a non-CD200 portion, wherein said non-CD200 portion is an IgG4 Fc fragment and comprises S228P, M428L and N434S mutations according to the EU numbering system and deletion of the first 5 amino acids of the hinge,
wherein Glycine 232 of the mutated CD200 portion is directly fused to the non-CD200 IgG4 Fc fragment at amino acid 6 according to the IMGT numbering system.

According to a further aspect of the invention, there is provided a polynucleotide encoding the fusion protein as defined herein.

According to yet further aspect, there is provided a pharmaceutical composition comprising the fusion protein as defined herein.

In another aspect of the invention, there is provided the fusion protein, polynucleotide, or pharmaceutical composition as defined herein for use in the preparation of a medicament. In another aspect of the invention, there is provided the fusion protein, polynucleotide, or pharmaceutical composition as defined herein for use in therapy. In another aspect of the invention, there is provided the fusion protein, polynucleotide, or pharmaceutical composition as defined herein for use in the treatment of an autoimmune disease, an allergic disease, a neurodegenerative disorder, an inflammatory disorder, Th2-induced airway inflammation, diabetic neuropathy, neurodegeneration, or neuropathic pain. In another aspect of the invention, there is provided the fusion protein, polynucleotide, or pharmaceutical composition as defined herein for use in treatment of rheumatoid arthritis, asthma, or atopic dermatitis. In another aspect of the invention, there is provided the fusion protein, polynucleotide, or pharmaceutical composition as defined herein for use in treatment of an autoimmune disease affecting a neuromuscular system, vascular system, eye, skin, digestive tract, lung, kidney, liver, peripheral or central nervous system, bone, cartilage or joints.

### BRIEF DESCRIPTION OF THE FIGURES

FIGS. 1A-1B: Sensorgrams of BIAcore assays showing the association and dissociation phases of human CD200R binding to a captured mutated CD200-Fc fusion protein (ARQ-234; FIG. 1A) or wild-type CD200-Fc (DS-155; FIG. 1B).

FIGS. 2A-2B: Sensorgrams of BIAcore assays showing the association and dissociation phases of cynomolgus CD200R1 binding to a captured mutated CD200-Fc fusion protein (ARQ-234; FIG. 2A) or wild-type CD200-Fc (DS-155; FIG. 2B).

FIG. 3: Bar graphs showing the inhibition of LPS stimulated IL-6 release from U937-CD200R cells following treatment with mutated fusion protein (ARQ-234), either in the presence of Fc block (top panel) or without Fc block (lower panel). The percent inhibition shown is relative to LPS stimulated IL-6 release from U937-CD200R cells without treatment with mutated or wild type fusion protein set at 0%.

FIG. 4: Binding of mutated CD200-Fc (ARQ-234) or wild-type CD200-Fc (DS-155) to U937-CD200R cells, visualised using a fluorescent anti-human secondary after 1, 4 and 24 hours.

FIG. 5: A) Schematic of the PK analysis protocol. B) Graph showing the concentration of mutated CD200 fusion protein (ARQ-234) measured in the serum of cynomolgus monkeys at the indicated time post-dosage (at time=0).

FIG. 6: Bar graphs showing the inhibition of IL-6 in response to dose titrations of DS-155 (wt CD200-Fc) and DS-192 (13 nM high affinity CD200-Fc).

FIG. 7: Bar graph showing the inhibition of TNFα by DS-192. Error bars represent standard deviation between biological replicates.

FIG. 8: Bar graphs showing the inhibition of ERK-phosphorylation by DS-155 and DS-192 measured by flow cytometry in permeabilized cells with an anti-pERK antibody and the presence of huCD200-Fc fusions.

FIG. 9: Line graph showing the clinical scores of paw arthritis in male DBA/1J mice measured from day 25-36 on alternate days. Data is represented as Mean±SEM. **p<0.01; ***p<0.001 vs Disease+Dexa, Disease+DS-198, & Disease+DS-227, Two-way RM ANOVA followed by Tukey's multiple comparisons test.

FIG. 10: Bar graph showing the change in ear thickness of a humanized mouse model of contact hypersensitivity from day 0. The values shown are the combined value for the right and left ear.

FIG. 11: Bar graph showing IL-1β cytokine levels in tissue homogenates of a humanized mouse model of contact hypersensitivity on day 15. Data is represented as Mean±SEM. †p<0.05, ††p<0.01 vs isotype control, unpaired Student's t-test; *p<0.05, **p<0.01 vs negative control, unpaired Student's t-test.

FIG. 12: Bar graph showing GM-CSF cytokine levels in tissue homogenates of a humanized mouse model of contact hypersensitivity on day 15. Data is represented as Mean±SEM. †p<0.05, ††p<0.01 vs isotype control, unpaired Student's t-test; *p<0.05, **p<0.01 vs negative control, unpaired Student's t-test.

FIG. 13: Bar graph showing IL-13 cytokine levels in tissue homogenates of a humanized mouse model of contact hypersensitivity on day 15. Data is represented as Mean±SEM. †p<0.05, ††p<0.01 vs isotype control, unpaired Student's t-test; *p<0.05, **p<0.01 vs negative control, unpaired Student's t-test.

FIG. 14: Schematic of ARQ-234 CD200-Fc fusion showing mutations for high affinity CD200R and FcRn binding.

FIGS. 15A-15F: (FIG. 15A) Graph showing 100 μg/mL antibody recognizing CD64 (FcγRI) Fc gamma receptor activity when co-incubated with varying doses of DS-192 (ha CD200-IgG4 Fc) in a U937 cell assay. (FIG. 15B) Graph showing 0 μg/mL antibody recognizing CD64 (FcγRI) Fc gamma receptor activity when co-incubated with varying doses of DS-192 (ha CD200-IgG4 Fc) in a U937 cell assay. (FIG. 15C) Graph showing 100 μg/mL antibody recognizing CD16 (FcγRIII) Fc gamma receptor activity when co-incubated with varying doses of DS-192 (ha CD200-IgG4 Fc) in a U937 cell assay. (FIG. 15D) Graph showing 0 μg/mL antibody recognizing CD16 (FcγRII) Fc gamma receptor activity when co-incubated with varying doses of DS-192 (ha CD200-IgG4 Fc) in a U937 cell assay. (FIG. 15E) Graph showing 100 μg/mL antibody recognizing CD32 (FcγRII) Fc gamma receptor activity when co-incubated with varying doses of DS-192 (ha CD200-IgG4 Fc) in a U937 cell assay. (FIG. 15F) Graph showing 0 μg/mL antibody recognizing CD32 (FcγRII) Fc gamma receptor activity when co-incubated with varying doses of DS-192 (ha CD200-IgG4 Fc) in a U937 cell assay.

### DETAILED DESCRIPTION OF THE INVENTION

According to a first aspect of the invention, there is provided a fusion protein comprising:
(i) a mutated CD200 portion comprising mutations at amino acid residue positions 130 and 131, wherein said mutations are K130Y and I131Y; and
(ii) a non-CD200 portion, wherein said non-CD200 portion is an IgG4 Fc fragment and comprises S228P, M428L and N434S mutations according to the EU numbering system and deletion of the first 5 amino acids of the hinge,
wherein Glycine 232 of the mutated CD200 portion is directly fused to the non-CD200 IgG4 Fc fragment at amino acid 6 according to the IMGT numbering system.

The inventors have found that mutations in the extracellular domain of CD200 at these amino acid residues produces a mutant CD200 portion with increased binding affinity to the CD200 receptor (CD200R). Furthermore, fusion proteins comprising the mutated CD200 portion as described herein have significant benefits, in particular in respect to providing treatment with greater clinical efficacy and at lower doses.

Therefore, in a particular embodiment, the fusion protein comprises the amino acid sequence of SEQ ID NO: 1. In a further embodiment, the fusion protein consists of the amino acid sequence of SEQ ID NO: 1. In a yet further embodiment, the fusion protein is ARQ-234.

SEQ ID NO: 1 (also referred to herein as “ARQ-234”) consists of the following sequence:

QVQVVTQDEREQLYTPASLKCSLQNAQEALIVTWQKKKAVSPENMVTFSENHGVVIQPAYK

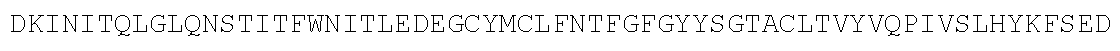

HLNITCSATARPAPMVFWKVPRSGIENSTVTLSHPNGTTSVTSILHIKDPKNQVGKEVICQVL

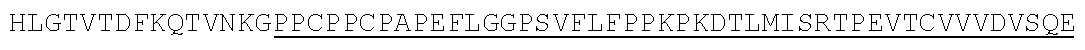

DPEVQFNWYVDGVEVHNAKTKPREEQFNSTYRVVSVLTVLHQDWLNGKEYKCKVSNKGL
PSSIEKTISKAKGQPREPQVYTLPPSQEEMTKNQVSLTCLVKGFYPSDIAVEWESNGQPEN

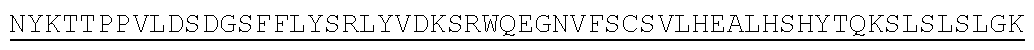

wherein the highlighted amino acids represent the positions of mutations relative to wild-type CD200 or IgG4 Fc and the underlined sequence represents the non-CD200 Fc fragment.

In one embodiment, ARQ-234 may further include an N-terminal signal sequence that is a human IgG heavy chain signal peptide. In a further embodiment, the N-terminal signal sequence comprises or consists of the amino acid sequence of SEQ ID NO. 3.

SEQ ID NO: 3 consists of the following sequence:

MEFGLSWLFLVAILKGVQC

The term “CD200 protein” as used herein, refers to wild-type CD200 protein.

The term “wild-type” as used herein, refers to proteins, peptides, amino acid and nucleotide sequences which are present in nature. For example, the term “wild-type CD200 protein” as used herein, refers to any full-length isoform of CD200 (UNIPROT P41217 OX2G_HUMAN) or any portion thereof (including naturally occurring protein polymorphisms) which binds to the CD200 receptor (CD200R). CD200 protein is also known as OX-2 membrane glycoprotein.

Wild-type CD200 is a cell surface protein, having an N-terminal extracellular domain, and short transmembrane and cytoplasmic domains. The extracellular domain binds to target receptors such as the CD200 receptor. In one embodiment, the CD200 protein is the extracellular domain of CD200, or any portion thereof, which binds to the CD200 receptor.

The term “position” as used herein, refers to the residue number in an amino acid sequence where 1 is the first translated amino acid. It will therefore be appreciated that the numbering of amino acid positions within the CD200 portion as defined herein is relative to the amino acid sequence including the N-terminal signal sequence representing the first 30 amino acids of the CD200 portion (as bolded in SEQ ID NO: 2).

The term “mutated” or “mutation” as used herein, refers to proteins, peptides, amino acid and nucleotide sequences which have undergone a change in their form from the wild-type equivalent to become a mutant. For example, a mutated or mutant protein may have undergone a change in the amino acid and/or nucleotide sequence when compared to the corresponding wild-type sequence, such a change may also be referred to as a mutation.

References herein to “mutated CD200 protein” and “mutated CD200 portion”, refer to full length CD200 protein or any portions thereof, which bind to the CD200 receptor, comprising a mutated amino acid residue or multiple mutated amino acid residues in the amino acid sequence so that it is similar but no longer identical to the wild-type CD200 protein. According to the first aspect of the invention as defined herein, the mutated CD200 portion comprises K130Y and I131Y mutations. Thus, in one embodiment, the mutations are substitution mutations.

In one embodiment, the fusion protein may be made synthetically or recombinantly. In a further embodiment, the fusion protein may be made synthetically. In an alternative embodiment, the fusion protein may be made recombinantly.

In one embodiment, the mutated CD200 portion binds to the CD200 receptor with greater affinity than wild-type CD200.

In one embodiment, the mutated CD200 protein/portion may include the entire extracellular domain of CD200 or portions thereof. In further embodiments, the mutated CD200 protein includes a signal sequence. It will be appreciated that secreted proteins comprise a number of amino acids at the N-terminus which make up a signal sequence which may be cleaved prior to secretion. Thus, in certain embodiments, the mutated CD200 portion comprises an N-terminal signal sequence. In one embodiment, the mutated CD200 portion includes a signal sequence at the N-terminus which is cleaved prior to secretion from the producing cell. In a further embodiment, the signal sequence comprises the first 30 amino acids of wild-type CD200 protein. In a yet further embodiment, the signal sequence represents the first 30 amino acids of the CD200 portion. In a yet further embodiment, the signal sequence is SEQ ID NO: 3. Thus, in certain embodiments, the fusion protein comprises a sequence as defined herein, where the amino acids which comprise the signal sequence are absent. For example, where amino acids 1-30 of wild-type CD200 protein are absent and the mutated CD200 protein comprises a sequence corresponding to amino acids 31-232 of SEQ ID NO: 2. Therefore, in a further embodiment, the fusion protein comprises the amino acid sequence of SEQ ID NO: 2. In a yet further embodiment, the fusion protein consists of the amino acid sequence of SEQ ID NO: 2. In a yet further embodiment, the fusion protein consists of the amino acid sequence of SEQ ID NO: 3 at the N-terminus of SEQ ID NO: 1.

SEQ ID NO: 2 consists of the following sequence:

MERLVIRMPFSHLSTYSLVWVMAAVVLCTAQVQVVTQDEREQLYTPASLKCSLQNAQEALI
VTWQKKKAVSPENMVTFSENHGVVIQPAYKDKINITQLGLQNSTITFWNITLEDEGCYMCLF

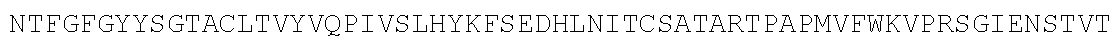

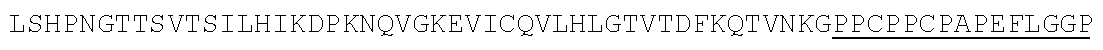

SVFLFPPKPKDTLMISRTPEVTCVVVDVSQEDPEVQFNWYVDGVEVHNAKTKPREEQFNS
TYRVVSVLTVLHQDWLNGKEYKCKVSNKGLPSSIEKTISKAKGQPREPQVYTLPPSQEEMT
KNQVSLTCLVKGFYPSDIAVEWESNGQPENNYKTTPPVLDSDGSFFLYSRLTVDKSRWQE

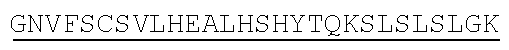

wherein the amino acids in bold represent the signal sequence, the highlighted amino acids represent the positions of mutations relative to wild-type CD200 or IgG4 Fc and the underlined sequence represents the non-CD200 Fc fragment. The present disclosure also includes the disclosed protein sequences, but lacking the C-terminal lysines, e.g., proteins in which the C-terminal lysine (K) has been cleaved during secretion from mammalian cells.

In one embodiment, the fusion protein is encoded by a polynucleotide of SEQ ID NO: 4. It is important to note that there is degeneracy of the genetic code, meaning that that most amino acids are specified by more than one codon. Thus, since numerous distinct codons define the same amino acid, more than one polynucleotide sequence can code for the same amino acid sequence. Therefore, SEQ ID NO: 4 represents one exemplary permutation of a polynucleotide sequence that can code for the fusion protein. Any permutations and combinations of all described elements in this application should be considered as disclosed by the description of the present application, unless the context indicates otherwise.

(SEQ ID NO: 4)
ATGGAACGGCTGGTCATCAGGATGCCCTTCAGCCACCTGTCTACCTACA
GCCTCGTGTGGGTTATGGCCGCCGTGGTTCTGTGTACAGCTCAGGTGCA
GGTCGTGACCCAGGATGAGAGAGAGCAGCTGTATACCCCTGCCAGCCTG
AAGTGTTCCCTGCAGAATGCCCAAGAGGCCCTGATCGTGACCTGGCAGA
AGAAGAAGGCTGTCTCCCCTGAGAACATGGTCACCTTCTCCGAGAATCA
CGGCGTCGTGATCCAGCCTGCCTACAAGGACAAGATCAACATCACACAG
CTGGGCCTGCAGAACTCCACCATCACCTTTTGGAACATCACCCTGGAAG
ATGAGGGCTGCTACATGTGCCTGTTCAACACCTTCGGCTTCGGCTACTA
CTCTGGCACCGCTTGTCTGACCGTGTACGTGCAGCCTATCGTGTCCCTG
CACTACAAGTTCTCCGAGGACCACCTGAATATCACCTGTTCCGCCACCG
CCAGACCTGCTCCTATGGTGTTTTGGAAGGTGCCCAGATCCGGCATCGA
GAACAGCACCGTGACACTGTCTCACCCTAACGGCACCACCTCCGTGACC
TCCATCCTGCACATCAAGGACCCCAAGAATCAAGTGGGCAAAGAAGTGA
TCTGTCAGGTTCTGCACCTGGGCACAGTGACCGACTTCAAGCAGACCGT
GAACAAGGGCCCTCCTTGTCCTCCATGTCCTGCTCCAGAATTTCTCGGC
GGACCCTCCGTGTTCCTGTTTCCTCCAAAGCCTAAGGACACCCTGATGA
TCTCTCGGACCCCTGAAGTGACCTGCGTGGTGGTGGATGTGTCTCAAGA
GGACCCCGAGGTGCAGTTCAATTGGTACGTGGACGGCGTGGAAGTGCAC
AACGCCAAGACCAAGCCTAGAGAGGAACAGTTCAACTCCACCTACAGAG
TGGTGTCCGTGCTGACCGTGCTGCACCAGGATTGGCTGAACGGCAAAGA
GTACAAGTGCAAGGTGTCCAACAAGGGACTGCCCTCCAGCATCGAAAAG
ACCATCTCCAAGGCTAAGGGCCAGCCTCGGGAACCTCAGGTTTACACCC
TGCCTCCAAGCCAAGAGGAAATGACCAAGAACCAGGTGTCCCTGACCTG
CCTGGTCAAGGGCTTCTACCCTTCCGACATTGCCGTGGAATGGGAGTCT
AACGGCCAGCCAGAGAACAACTACAAGACCACACCTCCTGTGCTGGACT
CCGACGGCTCCTTCTTTCTGTACTCTCGCCTGACCGTGGACAAGTCTAG
GTGGCAAGAGGGCAACGTGTTCTCCTGCTCTGTGCTGCACGAGGCCCTG
CACTCTCACTACACCCAGAAGTCCCTGTCTCTGTCTCTGGGCAAGTGAT
GA

The term “portion” as used herein with reference to proteins, peptides and amino acid and nucleotide sequences, refers to fragments and derivatives that are functional, i.e. bind to their target.

The term “fragment” as used herein refers to a part of a protein, peptide, amino acid or nucleotide sequence that recognises and binds its target, such as a receptor.

The term “derivatives of” and “mutant” as used herein, refer to a protein, peptide, amino acid or nucleotide sequence that shares at least 70% (such as 75%, 80%, 85%, 90%, 95% or 99%) sequence similarity with and functions like the wild-type equivalent. Thus, a mutant may be a derivative of a wild-type equivalent.

The term “amino acid residue” as used herein, refers to a monomeric unit in a polymeric chain, i.e. a single amino acid in a protein.

As shown by the data presented herein, the mutated CD200 proteins/portions of the invention bind more tightly to the CD200 receptor and exhibit longer residence time on the receptor than wild-type CD200 protein.

### Fusion Protein

According to the first aspect of the invention as defined herein, there is provided a fusion protein comprising the mutated CD200 protein/portion as defined herein fused to a non-CD200 portion.

The term “fusion protein” as used herein, refers to one or more amino acid sequences, peptides and/or proteins joined together using methods well known in the art and as described in, for example U.S. Pat. Nos. 5,434,131 and 5,637,481. The joined amino acid sequences, peptides or proteins thereby form one fusion protein.

In some embodiments, the mutated CD200 protein/portion defined herein is fused at the C-terminus to a non-CD200 portion. Thus, in one embodiment, the orientation of the fusion protein from N- to C-terminus is: mutated CD200 portion-non-CD200 Fc fragment. In a further embodiment, the orientation of the fusion protein is therefore: mutated CD200 portion-IgG4 Fc fragment. In another embodiment, the orientation of the fusion protein from N- to C-terminus is: signal sequence-mutated CD200 portion-non-CD200 Fc fragment. In a yet further embodiment, the orientation of the fusion protein is therefore: signal sequence-mutated CD200 portion-IgG4 Fc fragment.

The term “non-CD200 portion” as used herein, may refer to any molecule, peptide or protein that does not bind to the CD200 receptor and does not interfere with the binding of CD200 to its target. Examples include, but are not limited to, an immunoglobulin (lg) constant region or a portion thereof; or fusion proteins where the non-CD200 portion is a synthetic molecule, for example PEG.

In one embodiment, said non-CD200 portion is an antibody fragment. In a particular embodiment, said non-CD200 portion is an Fc fragment. Therefore, the mutated CD200 fusion protein as described herein may also be called a mutant CD200-Fc. In a further embodiment, the Fc fragment is mammalian derived, such as derived from a human or monkey, such as human C (gamma) 1 which includes the hinge, CH2 and CH3 regions. In particular, the Fc fragment comprises the hinge region. The Fc fragment provides the advantage of increasing the serum half-life of the mutated CD200 proteins of the invention, and additionally increases binding avidity and enables agonistic signalling, by dimerising the CD200 proteins. It will be understood by one skilled in the art that the Fc region may be mutated to reduce its effector functions (see for example, U.S. Pat. Nos. 5,637,481 and 6,132,992).

In one embodiment, the Fc fragment is an IgG4 Fc fragment.

In a further embodiment, the non-CD200 portion is an antibody Fc fragment which comprises mutation of one or more amino acid residue(s). Thus, in a particular embodiment, the non-CD200 portion is an IgG4 Fc fragment and comprises S228P, M428L and N434S mutations, wherein the position of said mutations is according to the EU numbering system. Therefore, in one embodiment, the non-CD200 Fc fragment is a S228P, M428L and N434S derivative of human IgG4. The S228P mutation prevents Fab-arm exchange in antibodies. Therefore, the presence of an S228P mutation in the Fc fragment described herein is likely to increase stability of the fusion protein both in vivo and in vitro, leading to improved therapeutic efficacy and improved manufacturability. The presence of M428L and N434S mutations (also known as an “LS variant”) increases the affinity of the Fc fragment for human neonatal Fc receptor (hFcRn) at pH 6 and results in increased serum half-life (Zalevsky et al. (2010) Nat. Biotechnol., 28(2): 157-159, doi: 10.1038/nbt.1601 and Ko et al. (2014) Nature, 514(7524): 642-645, doi: 10.1038/nature13612), such as increased serum half-life of the fusion protein defined herein comprising the mutated non-CD200 Fc fragment. In a yet further embodiment, the non-CD200 IgG4 Fc fragment comprises deletion of the first 5 amino acids, such as the first 5 amino acids of the hinge region of said IgG4 Fc fragment. Thus, in one embodiment, the non-CD200 portion is an IgG4 Fc fragment and comprises S228P, M428L and N434S mutations according to the EU numbering system and deletion of the first 5 amino acids of the hinge. In a further embodiment, the non-CD200 portion is an IgG4 Fc fragment and comprises S228P, M428L and N434S and deletion of the first 5 amino acids of the Fc hinge region.

In one embodiment, the fusion protein is formed by direct fusion of the mutated CD200 portion to the non-CD200 Fc fragment. Such fusion will therefore be appreciated to not comprise a linker sequence between the mutated CD200 portion and the non-CD200 Fc fragment. For example, amino acid Glycine 232 of the mutated CD200 portion may be directly fused to amino acid 1 of the Fc hinge region. In another embodiment, the fusion protein is formed by direct fusion of amino acid Glycine 232 of the mutated CD200 portion to amino acid 6 of the IgG4 Fc fragment (in this case the first 5 amino acids of the Fc hinge region are deleted as described hereinbefore). In a further embodiment, the direct fusion is of amino acid Glycine 232 of the mutated CD200 portion to amino acid 6 of the Fc hinge region of said IgG4 Fc fragment. Thus, in one embodiment, the Glycine 232 of the mutated CD200 portion is directly fused to the non-CD200 Fc fragment at amino acid 6 of the Fc hinge region. According to these embodiments, the position in the Fc fragment of said fusion is according to the IMGT numbering system. Such direct fusion of the mutated CD200 portion to amino acid 6 of the IgG4 Fc fragment hinge region increases the stability of the resulting fusion protein without affecting the potent binding to CD200R compared to fusion proteins comprising a linker sequence. This result is surprising in light of previously reported data for Fc fusion proteins containing linker sequences.

For the purpose of this description, the term “position” as used herein with respect to mutations within a non-CD200 portion when said non-CD200 portion is an Fc fragment, refers to the residue number in an amino acid sequence according to the EU numbering system. Therefore, it will be appreciated that a mutation residue position as quoted herein for an amino acid of an Fc fragment relates to its position according to the EU numbering system. It will be further appreciated that other numbering systems developed for the numbering of residues in Fc fragment sequences, such as Kabat, AHo, IMGT, Chothia and Martin (enhanced Chothia), may alternatively be utilised. When used herein with respect to the point at which the mutated CD200 portion is fused to the non-CD200 Fc fragment, “position” refers to the residue number within the Fc fragment according to the IMGT numbering system. It will therefore be appreciated that a residue position for an amino acid of an Fc fragment hinge relates to its position according to the IMGT numbering system. Thus, the numbering herein of mutations within an Fc fragment refers to the EU numbering system, and the numbering of hinge amino acids refers to the IMGT numbering system. In some embodiments, the Glycine 232 of the mutated CD200 portion is directly fused to the non-CD200 Fc fragment at amino acid 224 of the IgG4 heavy chain of the Fc fragment according to the EU numbering system.

The proteins of the present invention are preferably produced by recombinant DNA methods by inserting a nucleic acid sequence encoding the CD200-Fc fusion protein or any portion thereof into a recombinant expression vector and expressing the nucleic acid sequence in a recombinant expression system under conditions promoting expression. Therefore, in one embodiment, the polynucleotide encoding the fusion protein additionally comprises a vector, such as pCDNA 3.1. In one embodiment, the fusion protein is flanked by one or more restriction enzyme sites. In another embodiment, the nucleic acid sequence encoding the CD200-Fc fusion protein or any portion thereof is inserted into the recombinant expression vector using in-fusion cloning. Thus in a further embodiment, the nucleic acid encoding the CD200-Fc fusion protein or any portion thereof comprises nucleic acid sequences at its termini which are complementary to those at the termini of the linearised vector, such as an overlap between the CD200-Fc fusion protein-encoding nucleic acid and the vector of between 12 and 21 base pairs/nucleotides, e.g. an overlap of 15 base pairs or an overlap of 20 base pairs.

According to a further aspect of the invention, there is provided a polynucleotide encoding a fusion protein as defined herein. The present disclosure includes a polynucleotide encoding a protein as defined herein and use of such nucleic acids to produce the proteins and/or for therapeutic purposes. Such polynucleotides may include DNA and RNA molecules (e.g., mRNA, self-replicating RNA, self-amplifying mRNA, etc.) that encode a protein as defined herein. Nucleic acid sequences encoding the proteins provided by this invention can be assembled from cDNA fragments and short oligonucleotide linkers, or from a series of oligonucleotides, to provide a synthetic gene which is capable of being inserted in a recombinant expression vector and expressed in a recombinant transcriptional unit. In one embodiment, the polynucleotide encodes a fusion protein comprising the amino acid sequence of SEQ ID NO: 1. In a further embodiment, the polynucleotide encodes a fusion protein consisting of the amino acid sequence of SEQ ID NO: 1. In a yet further embodiment, the polynucleotide encodes ARQ-234. In a particular embodiment, the polynucleotide encodes a fusion protein comprising the amino acid sequence of SEQ ID NO: 2. In a still further embodiment, the polynucleotide encodes a fusion protein consisting of the amino acid sequence of SEQ ID NO: 2. An exemplary polynucleotide sequence is provided in SEQ ID NO: 4.

Recombinant expression vectors include synthetic or cDNA-derived nucleic acid fragments encoding mutated CD200 operably linked to suitable transcriptional or translational regulatory elements derived from mammalian, microbial, viral or insect genes. Such regulatory elements include a transcriptional promoter, an optional operator sequence to control transcription, a sequence encoding suitable mRNA ribosomal binding sites, and sequences which control the termination of transcription and translation. The ability to replicate in a host, usually conferred by an origin of replication, and a selection gene to facilitate recognition of transformants may additionally be incorporated.

### Therapeutic Uses

The invention has particular application in therapy because the interaction between the CD200 protein and the CD200 receptor is characterised by rapid dissociation (“off”) rates which results in low affinity of CD200 for the CD200 receptor. Therefore, increasing the affinity of mutant CD200 protein and fusion proteins comprising a portion thereof for the CD200 receptor as presented herein, can be used in the manufacture of pharmaceutical compositions with more potent properties.

Furthermore, manufacturing costs for recombinant proteins are high and the mutant CD200 protein/fusion protein comprising a portion thereof, having higher affinity, can be used in pharmaceutical compositions at significantly lower doses than wild-type or non-mutated CD200 protein to achieve a therapeutic effect. Use of the mutant CD200 protein/fusion protein comprising a portion thereof may therefore be more cost effective in addition to being more clinically effective.

According to a further aspect of the invention, there is provided a pharmaceutical composition comprising the fusion protein as defined herein. In one embodiment, the pharmaceutical composition comprises a fusion protein comprising the amino acid sequence of SEQ ID NO: 1. In a further embodiment, the pharmaceutical composition comprises a fusion protein consisting of the amino acid sequence of SEQ ID NO: 1. In a yet further embodiment, the pharmaceutical composition comprises ARQ-234.

In one embodiment, the mutated CD200 protein or fusion protein as defined herein is a modulator of the CD200 receptor. The term “modulator” as used herein, refers to a substance which results in a change, for example a modulator of a protein may result in an increase or decrease in the activity of said protein. In view of the properties of the mutated CD200 proteins and fusion proteins of the invention, they are believed to be agonists of the CD200 receptor and therefore find utility in the treatment of autoimmune disease. Therefore, in a further embodiment, the mutated CD200 protein or fusion protein as defined herein is an agonist of the CD200 receptor.

Thus, according to a further aspect of the invention, there is provided the fusion protein as defined herein or the pharmaceutical composition as defined herein for use in the treatment of autoimmune disease.

As used herein, the terms “autoimmune disease” or “autoimmune disorder” are used interchangeably and refer to undesirable conditions that arise from an inappropriate or unwanted immune reaction against self-cells and/or tissues or transplanted cells and/or tissues. The term “autoimmune disease” or “autoimmune disorder” is meant to include such conditions, whether they be mediated by humoral or cellular immune responses.

In an alternative embodiment, there is provided the fusion protein as defined herein or the pharmaceutical composition as defined herein for use in the treatment of an allergic disease. As used herein, the terms “allergy” or “allergic disease” are used interchangeably and refer to a T helper 2 (TH2)-driven disease that develops primarily from activity of TH2 cells. Examples of allergic diseases include chronic allergic disease (such as hay fever or allergic rhinitis), allergic contact dermatitis, seasonal allergies, anaphylaxis, food allergies, asthma and atopic dermatitis.

Fusion proteins comprising the mutant CD200 proteins/portions defined herein may deactivate activated immune cells with higher efficiency than fusion proteins comprising wild-type or non-mutated CD200 proteins.

In one embodiment, the autoimmune disease is selected from autoimmune diseases affecting the neuromuscular system, vascular system, eye, skin, digestive tract, lung, kidney, liver, peripheral or central nervous system, bone, cartilage or joints.

In a further embodiment, the autoimmune disease is one or more autoimmune diseases selected from: acute disseminated encephalomyelitis (ADEM); acute necrotizing hemorrhagic leukoencephalitis; Addison's disease; agammaglobulinemia; alopecia areata; amyloidosis; ankylosing spondylitis; anti-GBM/anti-TBM nephritis; antiphospholipid syndrome (APS); asthma, atopic dermatitis; Autoimmune angioedema; autoimmune aplastic anemia; autoimmune dysautonomia; autoimmune hepatitis; autoimmune hyperlipidemia; autoimmune immunodeficiency; autoimmune inner ear disease (AIED); autoimmune myocarditis; autoimmune oophoritis; autoimmune pancreatitis; autoimmune retinopathy; autoimmune thrombocytopenia purpura (ATP); autoimmune thyroid disease; autoimmune urticarial; axonal & neuronal neuropathies; Balo disease; Behcet's disease; bullous pemphigoid and related autoimmune blistering diseases; cardiomyopathy; Castleman disease; celiac disease (such as refractory celiac disease type II); Chagas disease; chronic idiopathic urticaria; chronic inflammatory demyelinating polyneuropathy (CIDP); chronic recurrent multifocal ostomyelitis (CRMO); chronic spontaneous urticaria; Churg-Strauss syndrome; cicatricial pemphigoid/benign mucosal pemphigoid; Crohn's disease; Cogans syndrome; cold agglutinin disease; congenital heart block; Coxsackie myocarditis; CREST disease; essential mixed cryoglobulinemia; demyelinating neuropathies; dermatitis herpetiformis; dermatomyositis; Devic's disease (neuromyelitis optica); diabetic neuropathy; discoid lupus; Dressler's syndrome; endometriosis; eosinophilic esophagitis; eosinophilic fasciitis; erythema nodosum; experimental allergic encephalomyelitis; Evans syndrome; fibrosing alveolitis; giant cell arteritis (temporal arteritis); giant cell myocarditis; glomerulonephritis; Goodpasture's syndrome; granulomatosis with polyangiitis (GPA) (formerly called Wegener's granulomatosis); graft-versus-host disease (GvHD); Graves' disease; Guillain-Barre syndrome; Hashimoto's encephalitis; Hashimoto's thyroiditis; hemolytic anemia; Henoch-Schonlein purpura; herpes gestationis; hypogammaglobulinemia; Hidradenitis supporativa (HS); idiopathic thrombocytopenia purpura (ITP); IgA nephropathy; IgG4-related sclerosing disease; immunoregulatory lipoproteins; inclusion body myositis; inflammatory bowel disorder (IBD); inflammatory skin disease; interstitial cystitis; juvenile arthritis; juvenile diabetes (type 1 diabetes); juvenile myositis; Kawasaki syndrome; Lambert-Eaton syndrome; leukocytoclastic vasculitis; lichen planus; lichen sclerosus; ligneous conjunctivitis; linear IgA disease (LAD); lupus (SLE); lyme disease, chronic; macrophage activation syndrome (MAS); mastocytosis; Meniere's disease; microscopic polyangiitis; mixed connective tissue disease (MCTD); Mooren's ulcer; Mucha-Habermann disease; multiple sclerosis; myasthenia gravis; myositis; narcolepsy; neuromyelitis optica (Devic's); neutropenia; ocular cicatricial pemphigoid; optic neuritis; palindromic rheumatism; PANDAS (Pediatric Autoimmune Neuropsychiatric Disorders Associated with Streptococcus); Palmoplantar pustulosis (PPP); paraneoplastic cerebellar degeneration; paroxysmal nocturnal hemoglobinuria (PNH); Parry Romberg syndrome; Parsonnage-Turner syndrome; pars planitis (peripheral uveitis); pemphigus; peripheral neuropathy; perivenous encephalomyelitis; pernicious anemia; POEMS syndrome; polyarteritis nodosa; type I, II, & III autoimmune polyglandular syndromes; polymyalgia rheumatic; polymyositis; postmyocardial infarction syndrome; postpericardiotomy syndrome; progesterone dermatitis; primary biliary cirrhosis; primary sclerosing cholangitis; psoriasis; psoriatic arthritis; idiopathic pulmonary fibrosis; pyoderma gangrenosum; pure red cell aplasia; Raynauds phenomenon; reactive arthritis; reflex sympathetic dystrophy; Reiter's syndrome; relapsing polychondritis; restless legs syndrome; retroperitoneal fibrosis; rheumatic fever; rheumatoid arthritis; sarcoidosis; Schmidt syndrome; scleritis; scleroderma; Sjogren's syndrome; sperm & testicular autoimmunity; stiff person syndrome; subacute bacterial endocarditis (SBE); Susac's syndroms; sympathetic ophthalmia; Takayasu's arteritis; temporal arteritis/giant cell arteritis; thrombocytopenia purpura (TTP); Tolosa-Hunt syndrome; transverse myelitis; type 1 diabetes; ulcerative colitis; undifferentiated connective tissue disease (UCTD); uveitis; vasculitis; vesiculobullous dermatosis; vitiligo; and Wegener's granulomatosis (now termed granulomatosis with polyangiitis (GPA).

In an alternative embodiment, there is provided the protein or fusion protein as defined herein or the composition as defined herein for use in the treatment of neurodegeneration.

In a further alternative embodiment, there is provided the protein or fusion protein as defined herein or the composition as defined herein for use in the treatment of neuropathic pain and inflammatory joint pain.

According to a further aspect of the invention, there is provided a method of treating an autoimmune disease, an allergic disease (e.g. rheumatoid arthritis, asthma, or atopic dermatitis), neurodegeneration, neuropathic pain, inflammatory joint pain, or diabetic neuropathy in a subject, comprising administering a fusion protein of the invention to a subject having at least one autoimmune disease, allergic disease, neurodegeneration, neuropathic pain, inflammatory joint pain, or diabetic neuropathy.

It will be appreciated that a protein or fusion protein of the invention can be administered as the sole therapeutic agent or it can be administered in combination therapy with one of more other compounds (or therapies) for the treatment of an autoimmune disease, an allergic disease (e.g. rheumatoid arthritis, asthma, or atopic dermatitis), neurodegeneration, neuropathic pain, inflammatory joint pain, or diabetic neuropathy.

Thus, according to a further aspect of the invention there is provided a pharmaceutical composition comprising a fusion protein as defined herein in combination with one or more additional therapeutic agents.

For the treatment of an autoimmune disease, an allergic disease (e.g. rheumatoid arthritis, asthma, or atopic dermatitis), neurodegeneration, neuropathic pain, inflammatory joint pain, or diabetic neuropathy the fusion protein of the invention may be advantageously employed in combination with one or more other medicinal agents, more particularly, with one or more immunosuppressive agents or adjuvants in immunosuppression therapy.

Examples of other therapeutic agents or treatments that may be administered together (whether concurrently or at different time intervals) with the compounds of the invention include but are not limited to: azathioprine; methotrexate; cyclosporine; monoclonal antibodies (e.g. basiliximab, daclizumab, and muromonab); and corticosteroids.

Each of the therapeutic agents present in the combinations of the invention may be given in individually varying dose schedules and via different routes. Additionally, the posology of each of the two or more agents may differ: each may be administered at the same time or at different times. A person skilled in the art would know through his or her common general knowledge the dosing regimens and combination therapies to use. For example, a protein or fusion protein of the invention may be used in combination with one or more other agents which are administered according to their existing combination regimen.

Generally, the proteins disclosed herein will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically acceptable adjuvants, if necessary to keep a polypeptide complex in suspension, may be chosen from thickeners such as carboxymethylcellulose, polyvinylpyrrolidone, gelatin and alginates.

The route of administration of pharmaceutical compositions according to the invention may be any of those commonly known to those of ordinary skill in the art. For therapy, including without limitation immunotherapy, the proteins of the invention can be administered to any patient in accordance with standard techniques. The administration can be by any appropriate mode, including parenterally, intravenously, intramuscularly, intraperitoneally, subcutaneously, transdermally, via the pulmonary route, for example, intranasally or inhaled, or also, appropriately, by direct infusion with a catheter, such as intracranially (e.g. i.c.v. into central nervous system ventricles or i.t. into the spinal cord). The dosage and frequency of administration will depend on the age, sex and condition of the patient, concurrent administration of other drugs, counterindications and other parameters to be taken into account by the clinician.

The proteins of the invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

It will be understood that all embodiments described herein may be applied to all aspects of the invention and vice versa.

Other features and advantages of the present invention will be apparent from the description provided herein. It should be understood, however, that the description and the specific examples while indicating preferred embodiments of the invention are given by way of illustration only, since various changes and modifications will become apparent to those skilled in the art. The following studies and protocols illustrate embodiments of the methods described herein.

### Example 1: Manufacture of Mutant and Wild Type CD200-Fc Molecules

### Gene Synthesis and Cloning

Gene synthesis (codon-optimized for CHO expression) of IgG4 S228P Fc, with mutations M428L+N434S, was carried out at GeneArt. These constructs were cloned into plasmid pCDNA 3.1 using in-fusion cloning to generate a vector backbone. To this backbone, codon-optimized DNA sequences encoding mutant or wild-type human CD200 residues 1-232 of UniProt P412178 (OX2G_Human), which includes an N-terminal signal sequence, were inserted N-terminal to IgG4 S228P Fc, to create direct fusions of amino acid Glycine 232 of CD200 to the Fc region. The sequences were confirmed bidirectionally.

### Giga Prep

Sequence confirmed plasmids were transformed in E. coli DH5a cells. A single colony of each target protein was selected and inoculated into 10.0 ml of LB containing ampicillin. Each mutant or wild-type construct was sub-cultured into 800 mL of Circlegrow media for the giga scale DNA preparation. The DNA was isolated using the Endotoxin Free Quanta Giga Kit.

### Protein Expression

The CD200-Fc proteins were generated by transient transfection of the expression plasmids into CHO-3E7 cells using Polyethyleneimine (PEI). Briefly, a 250 mL culture at 4.0×10⁶ cells/mL density, maintained at 37° C. in CD-Forti CHO medium, was transfected with 2 mg/L of plasmid using PEI at a 1:5 ratio. Twenty-four hours post transfection, cultures were shifted to 32° C., cells were fed with 10% feed C, glutamine, glucose and 0.5 M Sodium butyrate to enhance protein expression. The batch was monitored and supernatants containing overexpressed CD200-Fc were harvested at day 7 at a viability of ˜75%. Filtered supernatants were subjected to protein purification.

### Protein Purification

All the purification procedures were performed at 4° C. Culture harvests were loaded onto MabSelect SuRe affinity columns (5 mL), pre-equilibrated with 50 mM Sodium Phosphate, 150 mM NaCl pH 7.4, at a flowrate of 3 mL/min on a AKTA Pure platform. The column was washed with equilibration buffer and bound protein eluted using 20 mM sodium acetate, 150 mM NaCl pH 3.5. Elutes were neutralized with 10% v/v 1 M Tris pH 8.0 and analysed by SDS-PAGE. Fractions containing CD200-Fc were pooled and concentrated to 5 mL and subjected to gel-filtration chromatography (Hiload 16/600 Superdex-200 pg column) on a AKTA Pure platform. The protein was processed in 50 mM Sodium Phosphate, 150 mM NaCl pH 7.4 buffer system at 1.2 mL/min and collected in fractions. Fractions containing CD200-Fc dimer, as analysed by SDS-PAGE, were pooled and concentrated using an Amicon Ultra Centricon (10 kDa molecular weight cut-off) to 1.33 mg/mL (measured at UV 280 nm). The purified material was subjected to SEC-HPLC, LC-MS and EndoSafe LAL cartridge test to assess protein purity, molecular mass and endotoxin content, respectively. The final sample was stored at −80° C.

### Example 2: Binding Analysis of the Wild-Type and Mutant CD200-Fc Proteins

BIAcore experiments were performed by Syngene International Ltd. (Biocon Park, Plot No 2&3, Bommasandra Industrial Area, Bommasadra-Jigani Link Road, Bangalore—560099, India).

### Assay Principles

BIAcore instrumentation uses an optical method, Surface Plasmon Resonance (SPR), to measure the binding characteristics of two interacting molecules; in this case wild-type CD200-Fc or CD200-Fc mutants binding to the CD200 receptor (CD200R). The technique measures changes in the refractive index of one of the two interacting molecules captured on a chip (sensor) when the second molecule is flowed in solution over the captured partner. In these experiments CD200-Fc was immobilized on the chip (sensor) surface and CD200R was injected in an aqueous buffer over the captured CD200-Fc under continuous flow conditions. Changes in the CD200-Fc refractive index following CD200R binding were measured in real time and the result plotted as response units (RUs) versus time to generate sensorgrams.

### Instrumentation and Reagents

The experiments were performed on a GE Healthcare BIAcore T200. Human, cynomolgus and mouse CD200R proteins were purchased from Creative Biomart (CD200R1-320H, CD200R1-3483C, CD200R1-3280M). All measurements were performed in duplicate.

### Protocol

For human CD200 constructs: anti-human Fc (GE Healthcare) was covalently immobilized on a BIAcore CM5 sensor chip (Cytiva, BR100530) by amine coupling using a Cytiva kit (BR100839) following the manufacturer's instructions, targeting immobilization of 8,000-11,000 RU. CD200-Fc proteins were diluted to 0.5 μg/mL to 4 μg/mL in running Buffer (1×HBS-EP+pH7.4 (Cytiva BR100669), HEPES Buffered Saline pH 7.4 containing 3 mM EDTA and 0.05% v/v Surfactant P20) and flowed for 25 to 100 seconds at 10 L/min over the immobilized Anti-Human IgG Fc, with a stabilization time of 60 seconds. Between 35 and 250 RUs of CD200-Fc were captured, with higher RUs used for cynomolgus CD200R binding experiments. Human CD200R or Cynomolgus CD200R was serially diluted (3-fold dilutions) to 5 or more concentrations (depending on anticipated affinity) in running buffer along with buffer blank (0 nM), and flowed over the captured ligand at 30 to 50 μL/min flow rate for 120 seconds association followed by 120-360 seconds dissociation in running buffer. Analysis temperature was 25° C. This was followed by regeneration of the surface with 30-90 second pulse of 3M MgCl₂ flowed at 30 μL/min flow rate followed by stabilization of the surface with 60 second flow of running buffer.

For mouse CD200 constructs, muCD200R-Fc (Creative Biomart CD200R1-458M) diluted to 1 μg/mL in running buffer was flowed over the immobilized Anti-Human IgG Fc, with CD200 monomers serially diluted and flowed over captured CD200R. Other details were as above. Data analysis: experimental sensorgrams were analyzed in BIAevaluation software (GE Healthcare). The curves obtained were fitted to 1:1 Langmuir binding model by setting Rmax and RI as local parameters. Rate equations using standard parameters (e.g. ligand concentration, time) were used for iterative curve fitting. Closeness of fit was determined by algorithms provided by the manufacturer in the BIAevaluation software, and data accepted if the Chi² value was less than 10% of Rₘₐₓ, and the U value was less than or equal to 15.

Table 1 details the reagents used in developing and performing the assay.

TABLE 1
Reagents used in the course of the BIAcore experiments

| SI. No. | Product Description | Vendor | Catalogue No. |
|---|---|---|---|
| 1 | Human Antibody Capture Kit | Cytiva | BR100839 |
| 2 | Series S Sensor Chip CM5 | Cytiva | BR100530 |
| 3 | Recombinant HumanCD200R, | Creative | CD200R1-320H |
| | His-tagged | Biomart | |
| 4 | Recombinant CynoCD200R, | Creative | CD200R1-3483C |
| | His-tagged | Biomart | |
| 5 | Recombinant HumanCD200, | Creative | CD200-165H |
| | Fc-tagged | Biomart | |

### Results

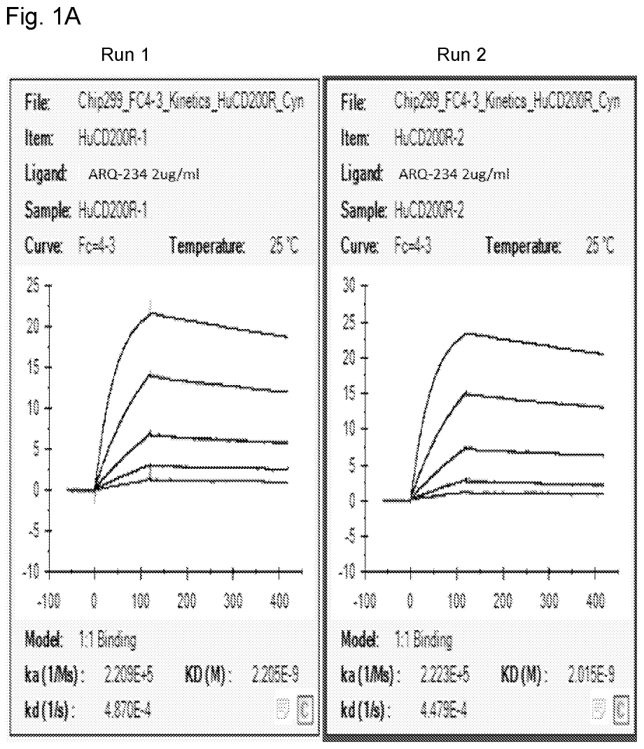

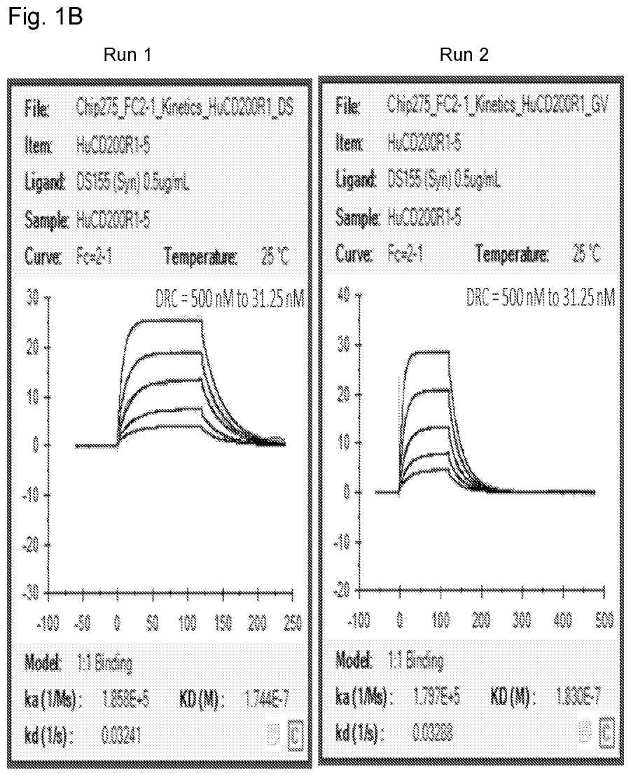

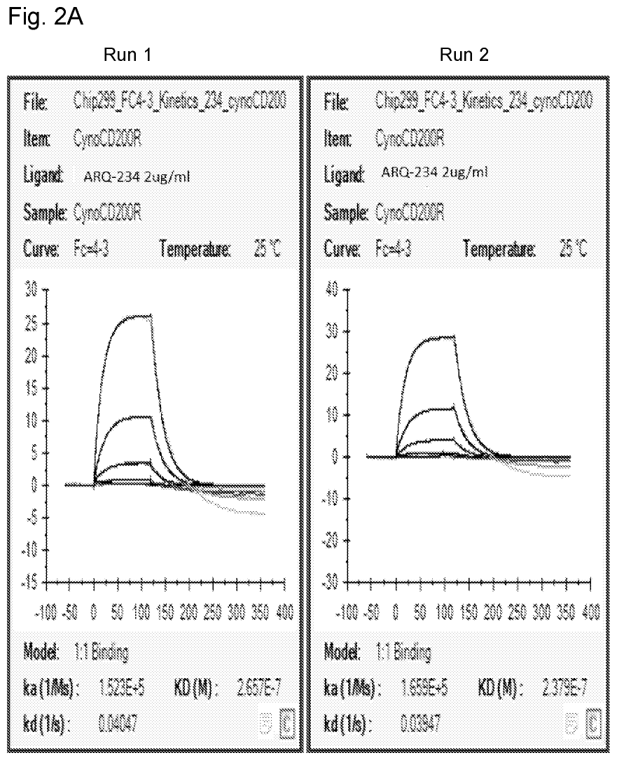

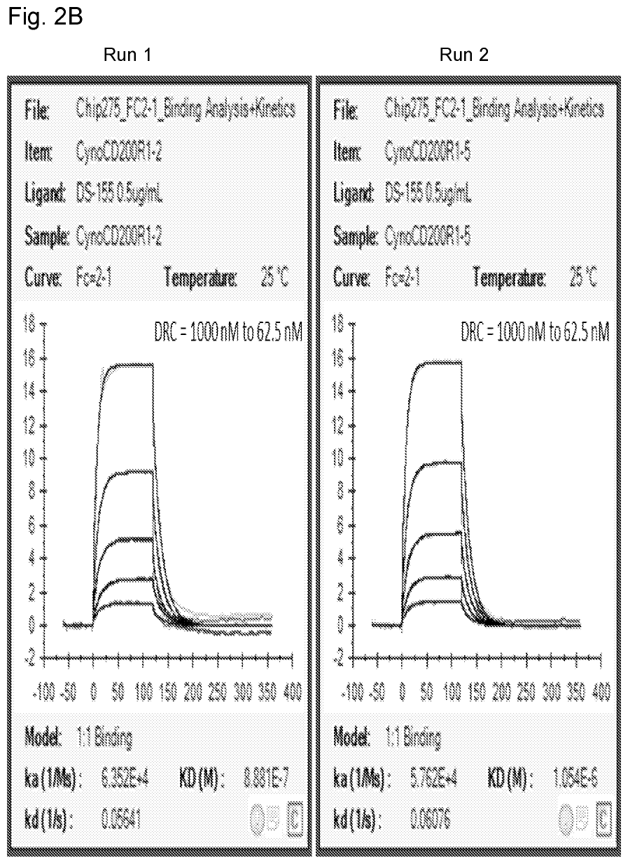

The results (Table 2 and FIGS. 1A-1B) show that the mutated CD200-Fc protein of the invention (ARQ-234) binds to the human CD200 receptor with approx. 84-fold greater affinity than wild-type CD200-Fc (DS-155). The tabulated off rates in Table 2 and the sensorgrams illustrated in FIGS. 1A-1B demonstrate an off rate for ARQ-234 and half-life on the receptor which are rates compatible with efficient agonism in functional cellular assays. Furthermore, the results in Table 3 and FIGS. 2A-2B show that ARQ-234 is able to bind cynomolgus CD200R, allowing this fusion protein to be evaluated in standard toxicology protocols.

TABLE 2
Surface Plasmon Resonance (SPR) affinity (K_{D}) and kinetic parameters (kₐ, k_{D},
t½) of wild-type and mutated CD200-Fc fusion molecules for human CD200R

| ID | Mutation | Fc | ka (1/Ms)* | kd (1/s)* | KD (nM)* | t½ (s) |
|---|---|---|---|---|---|---|
| DS-155 | Wild-Type | IgG4 S228P | 1.83E+05 | 3.26E−02 | 178.7 | 21 |
| ARQ- | K130Y, | IgG4 S228P, | 2.216E+05 | 4.675E−04 | 2.11 | 1476 |
| 234 | I131Y | M428L, N434S | | | | |

TABLE 3
Surface Plasmon Resonance (SPR) affinity (K_{D}) and kinetic parameters (kₐ, k_{D},
t½) of wild-type and mutated CD200-Fc fusion molecules for cyno CD200R

| ID | Mutation | Fc | ka (1/Ms)* | kd (1/s)* | KD (nM)* | t½ (s) |
|---|---|---|---|---|---|---|
| DS-155 | Wild-Type | IgG4 S228P | 6.06E+04 | 0.05859 | 971 | 12 |
| ARQ- | K130Y, | IgG4 S228P, | 1.591E+05 | 0.03997 | 251.8 | 17 |
| 234 | I131Y | M428L, N434S | | | | |

*ka (1/Ms), kd (1/s) and KD (nM) in both Table 2 and Table 3 are mean values of two runs.

TABLE 4
Mutant CD200 Fusion Proteins

| | | Mutations | |
|---|---|---|---|
| Construct | Species | (CD200) | Fc |
| DS-155 | Human | wt | IgG4 S228P |
| DS-192 | Human | K130Y | IgG4 S228P |
| ARQ-234 | Human | K130Y, | IgG4 S228P, |
| | | I131Y | M428L, N434S |
| DS-198 | Mouse | wt | muIgG2a |
| DS-227 | Mouse | H82Y, | muIgG2a |
| | | T125I | |

### Example 3: Cell Binding and Cell Activation Assays of the Wild-Type and Mutant CD200-Fc Proteins

### Assay Principles

To demonstrate the agonist activity of ARQ-234, the human monocyte cell line U937 (ATCC, CRL1539) was transfected with the cDNA for human CD200R. Cytokine production, including IL-6, from these cells can be induced by stimulation with PMA and then LPS.

### Cell Line Construction

The full length human CD200R gene, including the signal sequence, was cloned into pCDH-EF1-human CD200R-IRES-Puro lentivector (System Biosciences) downstream of the EF1α promoter. Lentiviral particles containing the expression construct were produced in 293TN producer cells and concentrated using PEG-it reagent (System Biosciences) according to the manufacturer's instructions.

The U937 human monocyte immortalised cell line was transduced with the lentiviral particles, with a range of MOls from 5 to 200, using the TransDux and Max Enhancer reagents (System Biosciences) according to the manufacturer's instructions. Transduced U937 cells were a) selected using puromycin (having first optimised puromycin concentration) and b) sorted by flow cytometry, to produce a stable, polyclonal CD200R-expressing line. Expression of CD200R was confirmed by Western blot in addition to flow cytometry.

### Cytokine Release (IL-6 Inhibition) Assay

50,000 U937 cells per well were seeded in 96 well plates and differentiated following incubation for 72 hours with 100 nM PMA. Following differentiation, the PMA containing media was replaced with fresh assay media and incubated for a further 2 hours prior to treatment. CD200-Fc constructs were added with or without Fc block to the cell culture and incubated for 1 hour, then cells were stimulated with 100 ng/ml LPS and incubated for a further 24 hours. Following the final incubation, cell supernatant (diluted 1:10) was collected and assayed for IL-6 secretion by ELISA assay using a commercially available kit.

### Cell Binding

U937 cells were re-suspended with a density of 0.1 million cells per test with 50 μL FACS buffer (1×PBS+2% FBS).

Treatment with CD200-Fc proteins (50 μL) was performed starting from 10 μg/mL with 3-fold dilutions up to 10 concentrations with FACS buffer and incubated for 1-hour, 4-hours and 24-hours at 37° C. At the end of each time point, cells were collected and washed. 10 μg/mL of anti-human secondary antibody was added and incubated for 30 minutes at 4° C. Cells were washed post incubation and stained to check viability (1 μL dye per million cells per mL 1×PBS) for 20 minutes at 4° C. Cells were washed and fixed with Fixation buffer (100 μL per test) at 4° C. for 20 minutes. Post incubation, cells were washed and the pellet was re-suspended in FACS buffer (100 μL per test) for data acquisition on the flow cytometer.

For the 24-hour time point, treatment with CD200-Fc protein was performed with cell culture media. Wash step=addition of 200 μL FACS buffer and centrifugation at 1400 RPM.

TABLE 5
Reagents used for cell binding assay

| SI. no. | Reagent | Make | Catalogue no. |
|---|---|---|---|
| 1. | Human BD Fc block | BD Biosciences | 564220 |
| 2. | Violet cell proliferation dye | ThermoFisher | C34557A |
| | | Scientific | |
| 3. | Goat anti-human IgG | Abcam | Ab98596 |
| | secondary antibody | | |
| 4. | Fixation buffer | BD Biosciences | 554655 |
| 5. | RPMI 1640 medium | Gibco | A1049101 |
| | (ATCC modification) | | |

### Results

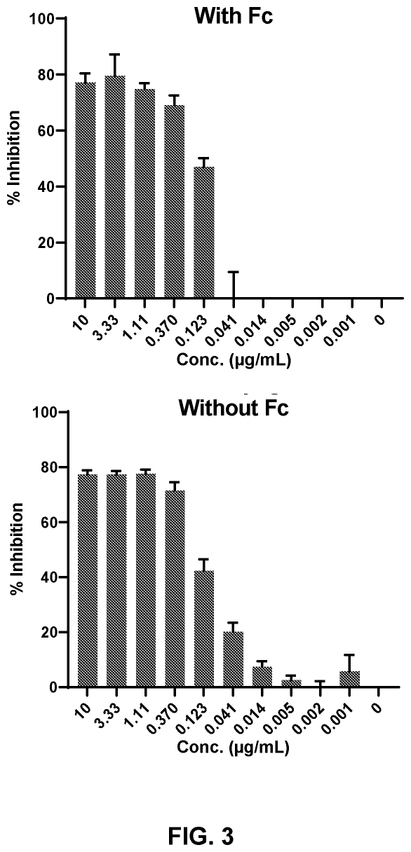

The data shown in FIG. 3 demonstrate that ARQ-234 is able to inhibit LPS stimulated IL-6 secretion in a concentration dependent manner. No significant difference in inhibition was observed in the presence of Fc block reagent in vitro (FIG. 3, top panel). While this is surprising as the IgG4 Fc domain of ARQ-234 binds to Fc gamma receptors and this should increase the avidity of the interaction with CD200R, this mechanism may still increase the potency of CD200-Fc proteins in vivo.

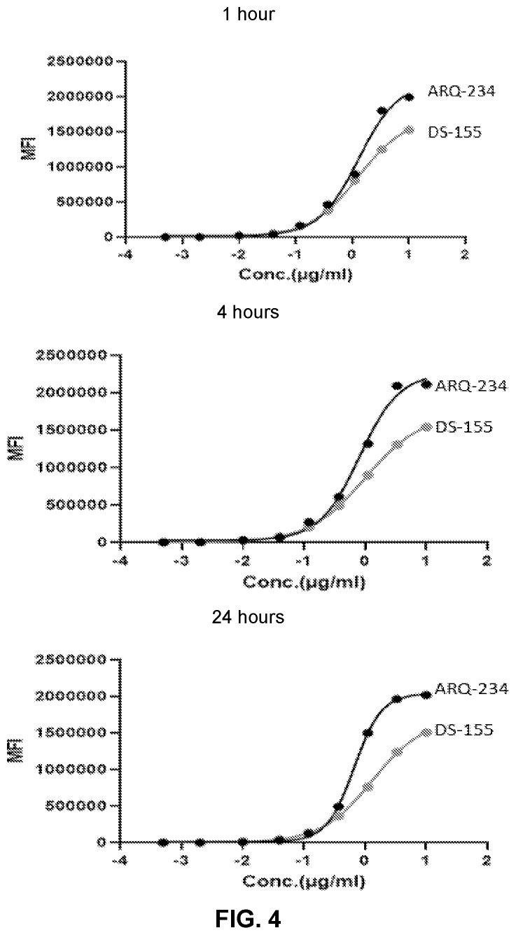

FIG. 4 shows the binding of the wild-type DS-155 and mutant ARQ-234 CD200-Fc proteins to CD200R-expressing U937 cells. This data demonstrates the superior binding of ARQ-234 to CD200R-expressing cells compared to DS-155 (wild-type CD200-Fc protein) at all time points.

### Example 4: PK Study of ARQ-234 in Serum of Cynomolgus Monkeys

### Protocol

2 cynomolgus monkeys per group (one male, one female) were dosed at 5 mg/kg with an i.v. bolus of protein at time 0. Blood samples were taken for PK analysis at the following timepoints: pre-dose, 0.25 hr, 0.5 hr, 1 hr, 4 hr, 8 hr, 24 hr, day 3, day 5, day 7, day 10, day 12, day 14, day 21, day 28.

### Serum Sample for PK Analysis

At least 0.8 mL blood samples were collected from a cephalic or saphenous vein at sampling time points from the two animals. For samples collected within the first hour of dosing, a ±1 minute deviation in sample collection time was acceptable. For the remaining time points, samples that were taken within 5% of the scheduled time are acceptable. All blood samples were collected into commercially available tubes containing coagulant. The tubes containing blood samples remained at room temperature for 30 minutes before centrifugation. The samples were centrifuged at 4° C. for 10 minutes at 1500×g within one hour of collection. About 400 μL serum per time point was collected post centrifugation. The samples were then quickly frozen over dry ice and kept at −60° C. or lower until transferred in dry ice for analysis. All samples were uniquely identified to indicate origin and collection time.

### Determination of Protein Concentration in Serum

The concentrations of analyte in serum were determined using a bioanalytical ELISA method. 96-well ELISA plates were coated overnight at 4° C. with 1 μg/ml Goat anti-Human IgG in Carbonate-bicarbonate buffer. After wash and blocking, serial diluted plasma samples were added and then biotin labeled Goat anti-human IgG (0.0625 ug/mL) was used as detection antibody. HRP-Streptavidin and TMB substrate were used for colour development. The reaction was stopped after approximately 5-10 minutes through the addition of 2M HCl. The absorbance was read at 450 nm and 540 nm using a microplate spectrophotometer (SpectraMax® M5e). The OD value of the samples were substituted into the standard curve to obtain the plasma concentration. The detection limit of this ELISA method LLOQ for Fc+Fc is 1 ng/mL.

The serum concentration of ARQ-234 in monkeys was subjected to a non-compartmental pharmacokinetic analysis by using the Phoenix WinNonlin software (version 8.1, Pharsight, Mountain View, CA). The linear/log trapezoidal rule was applied in obtaining the PK parameters.

### Results

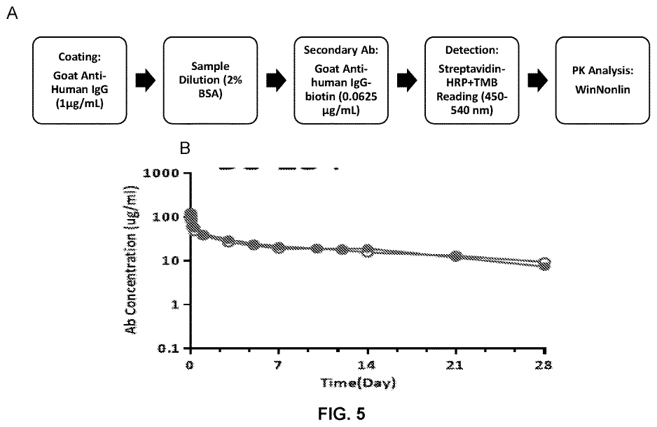

The data shown in Table 6 and FIG. 5 shows good serum stability of ARQ-234, with an average half-life in the two animals tested of 370.5 hours.

TABLE 6
PK parameters

| Animal No. | #3001 (M) | #3002 (F) |
|---|---|---|
| t_{1/2} (h) | 298 | 443\hat{ } |
| Cₘₐₓ (μg/ml) | 127 | 124 |
| AUC 0-t (h*μg/ml) | 12927 | 12259 |
| Cl_obs (ml/day/kg) | 7.45 | 6.56 |
| MRTINF_obs (h) | 415 | 607 |
| Vss_obs (mL/kg) | 129 | 166 |

\hat{ }The half-life was not accurate when the AUC_%Extrap_obs is greater than 20% or the Rsq_adjusted is less than 0.9.
The following parameters were tested in Table 6: half life (T_{1/2}); maximum serum concentration (Cₘₐₓ); area under the serum concentration from time zero to time t (28 days) (AUC 0-t); measured clearance rate (Cl_obs); mean residence time extrapolated to infinity (MRTINF); volume of distribution (Vss_obs). The half-life was calculated without data less than 1% of Cmax.

### Example 5: In Vitro Proof-of-Concept for High Affinity Murine CD200-Fc

Due to the lack of cross reactivity with murine CD200R, a mouse CD200-CD200R1 in silico model was generated, based on a published crystal structure, to engineer high affinity surrogate CD200-Fc proteins for proof-of-concept experiments in murine models of autoimmunity.

### Protocol

Murine CD200 constructs used Uniprot sequence O54901, containing the signal peptide and extracellular domains. Mutation numbers refer to the full Uniprot sequences including signal peptide.

Proteins were generated by transient transfection of pcDNA 3.1-based expression plasmids into CHO-3E7 cells using Polyethyleneimine (PEI). 24 hours post transfection, cultures were shifted to 32° C., fed with 10% feed C, glutamine, glucose and 0.5 M Sodium butyrate; supernatants were harvested and filtered at day 7. Purification was performed at 4° C. using MabSelect SuRe 5 ml affinity columns (Cytiva), pre-equilibrated with 50 mM Sodium Phosphate, 150 mM NaCl PH 7.4, at a flowrate of 3 mL/min on a AKTA Pure platform. The column was washed with equilibration buffer and bound protein eluted using 20 mM sodium acetate, 150 mM NaCl pH 3.5. Elutes were neutralized with 10% v/v 1 M Tris pH 8.0 and analysed by SDS-PAGE. Pooled fractions were concentrated to 5 mL and subjected to gel-filtration chromatography (Hiload 16/600 Superdex-200 pg column) on a AKTA Pure platform. The protein was processed in 50 mM Sodium Phosphate, 150 mM NaCl pH 7.4 buffer system at 1.2 mL/min. Fractions containing protein were pooled and concentrated using an Amicon Ultra Centricon (10 kDa molecular weight cut-off) to 1.33 mg/mL (measured at UV 280 nm). The purified material was subjected to SEC-HPLC, LC-MS and EndoSafe LAL cartridge test to assess protein purity, molecular mass and endotoxin content, respectively. Mouse CD200-his proteins were purified with Ni-NTA agarose resin using standard methodology. All proteins were stored at −80° C.

### Results

As shown in Table 7, the binding affinity of combination variant H82Y, T1251 is 43 nM, approximately 14-fold higher than wild type constructs contained a murine IgG2a Fc domain.

TABLE 7
Murine CD200 Variants

| Mutations | K_{D} | T_{1/2} (s) |
|---|---|---|
| wt | 584.0 ± 56.6 | 17.3 |
| H82Y, T125I | 43.3 ± 4.9 | 117.6 |

### Example 6: In Vitro Proof-of-Concept for High Affinity Human CD200-Fc

Human CD200-Fc (huCD200-Fc) proteins identified were tested for their ability to inhibit cytokine release from LPS-activated pro-monocytic, human myeloid leukemia cells (U937) engineered to express high levels of human CD200R.

### Protocol

Cell line construction, cytokine inhibition testing, and cell binding testing were conducted using similar techniques as in Example 3 above.

To test the ability of huCD200-Fc proteins to inhibit ERK-phosphorylation, To test the ability of huCD200-Fc proteins to inhibit ERK-phosphorylation, U937-CD200R cells were induced with PMA for 20 mins, and inhibition of ERK-phosphorylation by DS-155, DS-192 and DS-118 measured by flow cytometry in permeabilized cells with an anti-pERK antibody.

### Results

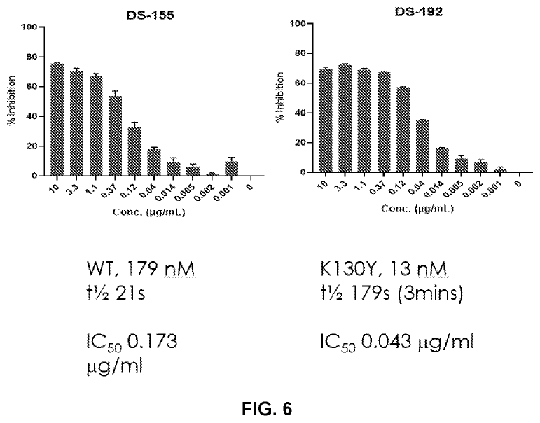

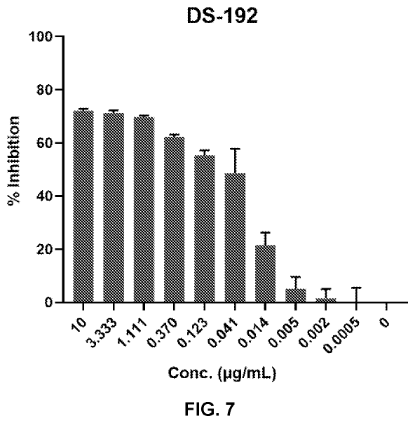

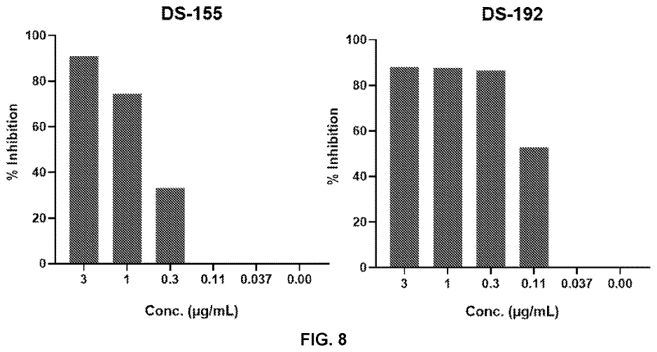

As shown in FIG. 6, higher affinity was observed for 13 nM DS-192, which exhibits more potent inhibition of IL-6 release than wild type DS-155. As shown in FIG. 7, inhibition TNF-α was also observed. As shown in FIG. 8, inhibition of ERK phosphorylation correlates with CD200 affinity.

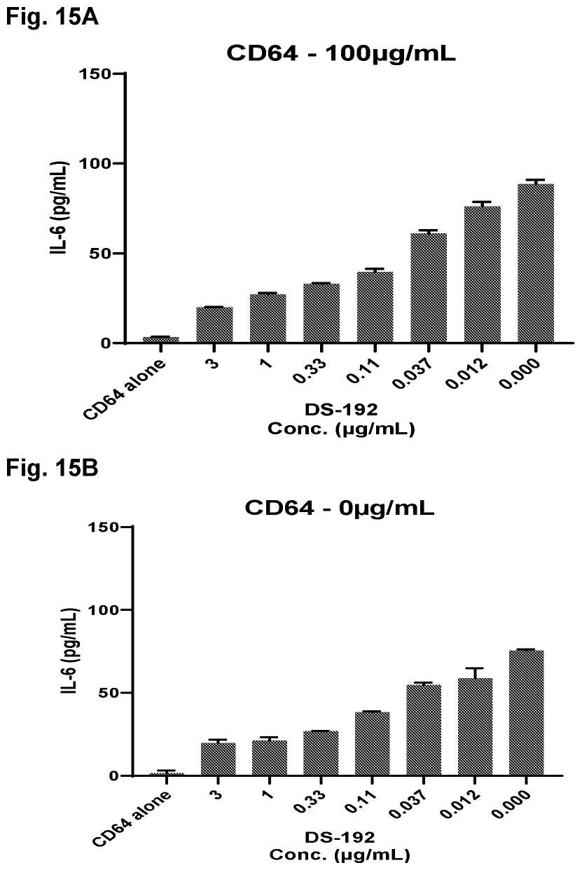

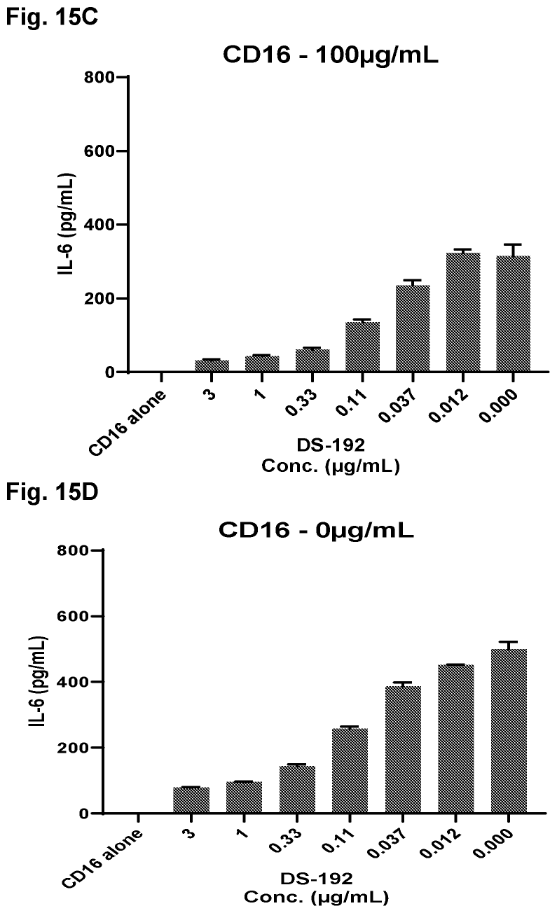

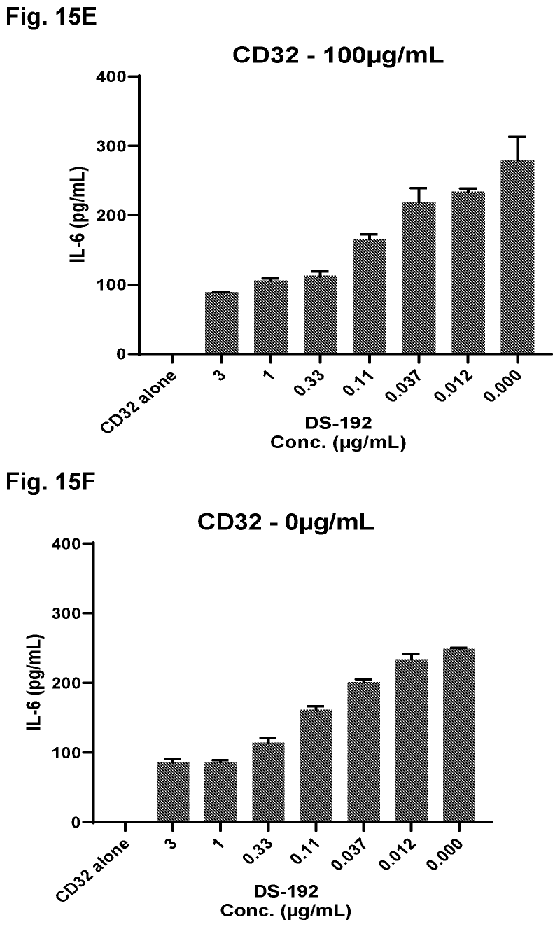

As shown in FIGS. 15A-15F, antibodies recognizing Fc gamma receptors do not inhibit the activity of DS-192 in vitro, which suggest that the Fc domain does not play a significant role in the inhibitory activity in this particular assay.

### Example 7: In Vivo Proof-of-Concept for High Affinity Murine CD200-Fc

A mouse model was used to show that higher affinity murine CD200-Fc protein decreases the clinical score in a mouse collagen-induced arthritis (CIA) model, with preventative dosing.

Mice possess four potential CD200 receptors, CD200R1-CD200R4, at least one of which may be activating; CD200R1 is the homologue of human CD200R. Knockout of either CD200 or CD200R1 in transgenic mice exacerbates or induces early onset in models of many autoimmune conditions, for example alopecia, arthritis, IBD25 and uveoretinitis.

CD200R agonism in rodent models, with patient samples in vitro, is known in the art and had previously been achieved with CD200-Fc fusion proteins, which suggested that a human CD200-Fc fusion protein could be used as a therapy for inflammatory disease. In common with other cell surface immune receptors, the affinity of CD200 for CD200R is low (in the high nanomolar range), so the ideal human therapeutic requires affinity enhancement for optimal potency. The Fc domain imparts an antibody-like serum half-life, and the dimeric format increases binding avidity and enables receptor cross-linking. Animal model data indicated that the sequence of the Fc domain was associated with murine IgG2a Fc fusions having optimal efficacy, likely by binding to Fc gamma receptors to facilitate the formation of cell-cell interactions, to further increase avidity. Antibody-dependent cellular cytotoxicity may also contribute, by removal of CD200R1 expressing cells. Therefore, an in vivo murine model was used to test the potency of a murine version of a high affinity CD200-Fc protein compared to wildtype CD200-Fc proteins.

### Protocol

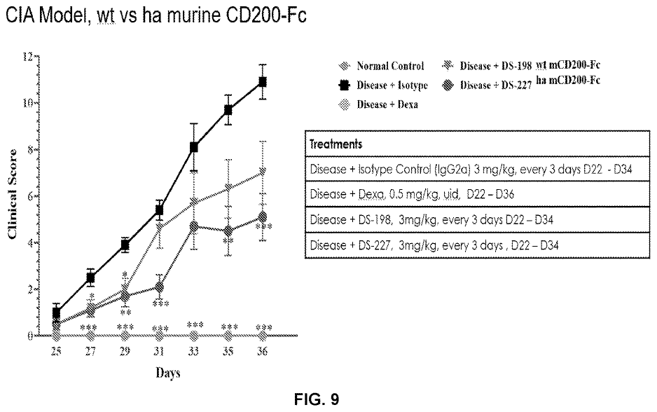

Wild type (DS-198) and higher affinity (DS-227) murine CD200-Fc proteins were tested using a CIA model by initiating dosing just prior to symptom onset. Arthritis was induced in male DBA/1J mice by intradermal injection of bovine type II collagen in CFA (complete Freund's adjuvant) on day 1, followed by a booster injection in incomplete Freund's adjuvant on day 21. On day 22, animals were randomized based on body weight, and injected once every 3 days until day 36 with 3 mg/kg murine IgG2a isotype control antibody, DS-198 (wild type muCD200-Fc) or DS-227 (high affinity 43 nM muCD200-Fc); the positive control group received oral 0.5 mg/kg dexamethasone dosed daily. Clinical scores of paw arthritis (blinded assessment) were measured from day 25-36 on alternate days. The data in FIG. 9 are shown as Mean±SEM. **p<0.01; ***p<0.001 vs Disease+Dexa, Disease+DS-198, & Disease+DS-227. Two-way RM ANOVA followed by Tukey's multiple comparisons test.

### Results

As shown in FIG. 9, the higher affinity CD200-Fc, DS-227, was significantly more potent in reducing clinical score than wild type (DS-198), at the selected dose of 3 mg/kg.

### Example 8: Proof of Concept Study Using High Affinity DS-192

Based on the results of the in vivo proof-of-concept for high affinity murine CD200-Fc study (CIA mouse study) described above, an in vivo proof-of-concept study was conducted to test DS-192, a high affinity human CD200-Fc fusion protein. A humanized model of oxazolone-induced contact hypersensitivity was designed using NOG-EXL mice, which could be engrafted with both human lymphocytes and myeloid cells.

### Protocol

Female NOG-EXL mice were engrafted with human cells, and randomized on the basis of % CD45+ cells aged week 20-21 (Day-1). On day 0 mice were sensitized with abdominal application of oxazolone (100 μL of 3% w/v oxazolone in acetone: alcohol 1:4), and challenged on days 5, 10 and 14 with topical application of 20 μL 2% w/v oxazolone (acetone: alcohol 1:4) to each ear (10 μL/side). Pre-sensitized huNOG-EXL mice underwent repeated oxazolone challenge on one ear, with DS-192 (huCD200-Fc, 13 nM) or a CD200R agonist antibody (CD200R mAb) dosed on the same day as each challenge. Isotype control antibody, CD200R agonist antibody and high affinity huCD200-Fc (DS-192) were dosed intravenously at 3 mg/kg on days 5, 10 and 14, 4 hours before oxazolone challenge. Ear thickness was measured just prior to challenge and 24 hours after each challenge, and on day 15 punch biopsies were taken for cytokine analysis by multiplex.

### Results

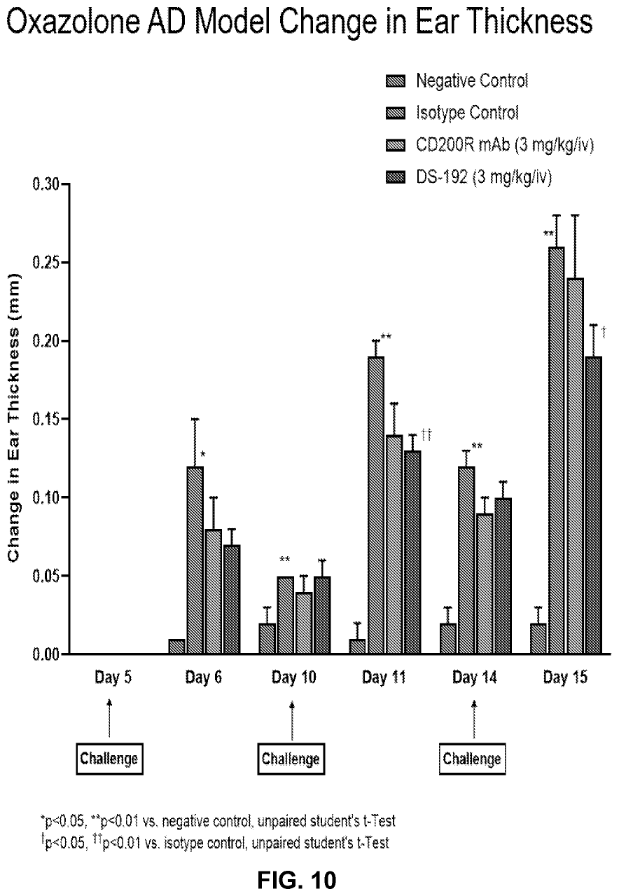

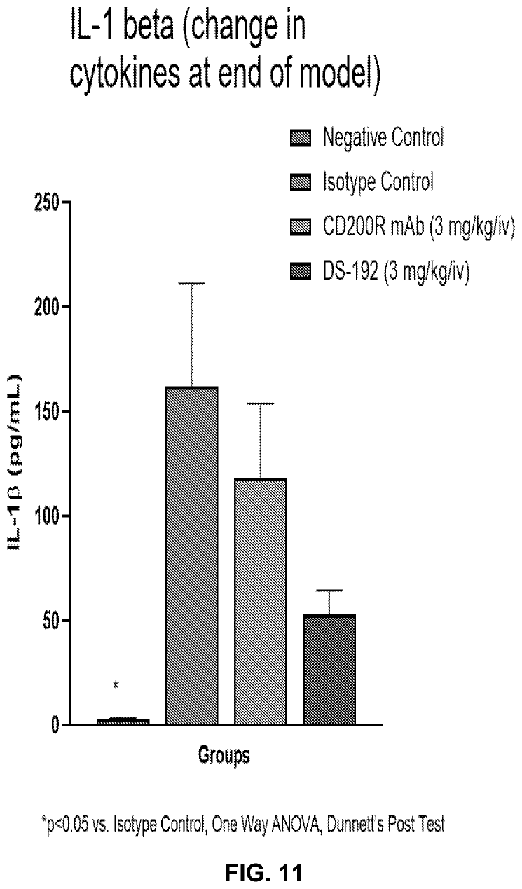

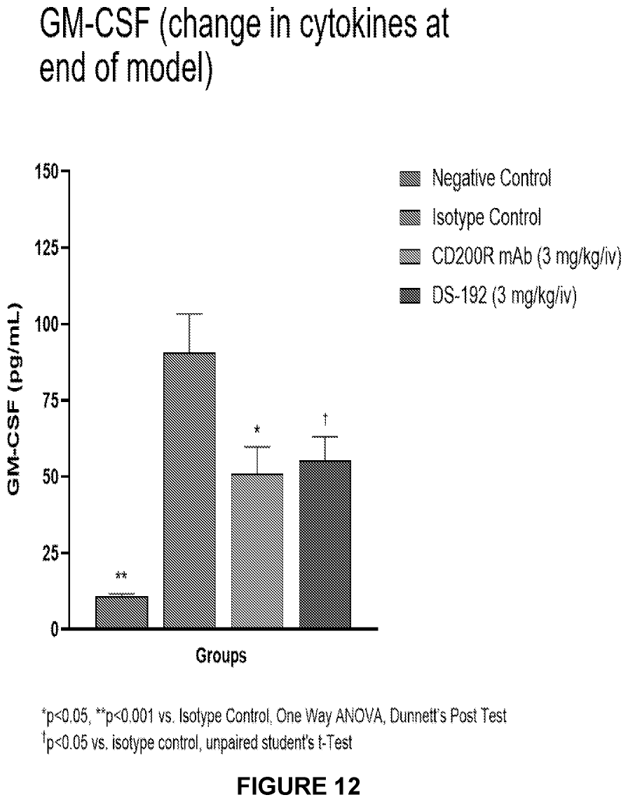

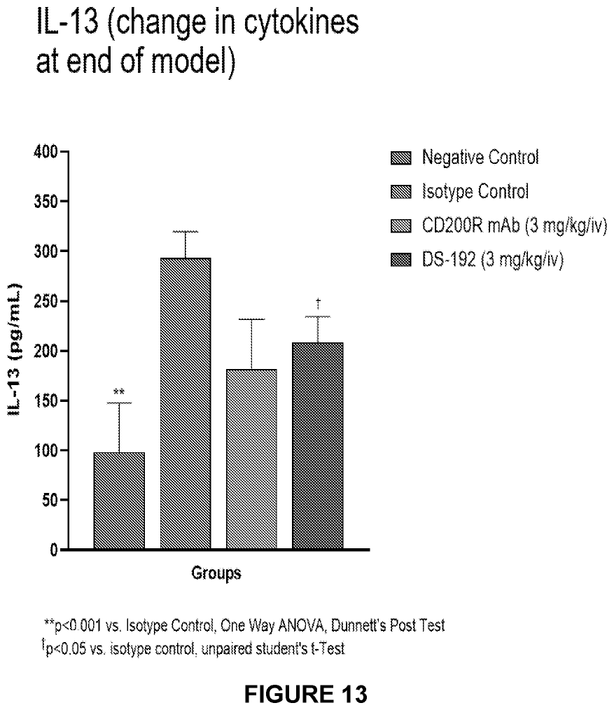

As shown in FIG. 10, the change in ear thickness (a surrogate for inflammatory response) was significantly reduced by DS-192 on the day after the 2^{nd} and 3^{rd} challenge compared to isotype control, in contrast to CD200R mAb which did not result in a significant decrease. Additionally, as shown in FIGS. 11, 12 and 13, a significant decrease in IL-1β, GM-CSF and IL-13 in ear tissue was observed at the end of the study in DS-192-treated mice. Therefore, the results showed that high affinity CD200-Fc has superior potency compared to a CD200R mAb in a humanized mouse model of contact hypersensitivity. As DS-192 has significantly lower CD200R affinity than ARQ-234, extrapolation of these advantageous indicates greater efficacy when using ARQ-234 for treating allergic diseases and skin inflammatory disorders.

### Example 9: Cell Assay Binding Analysis of High Affinity CD200-Fc Molecules

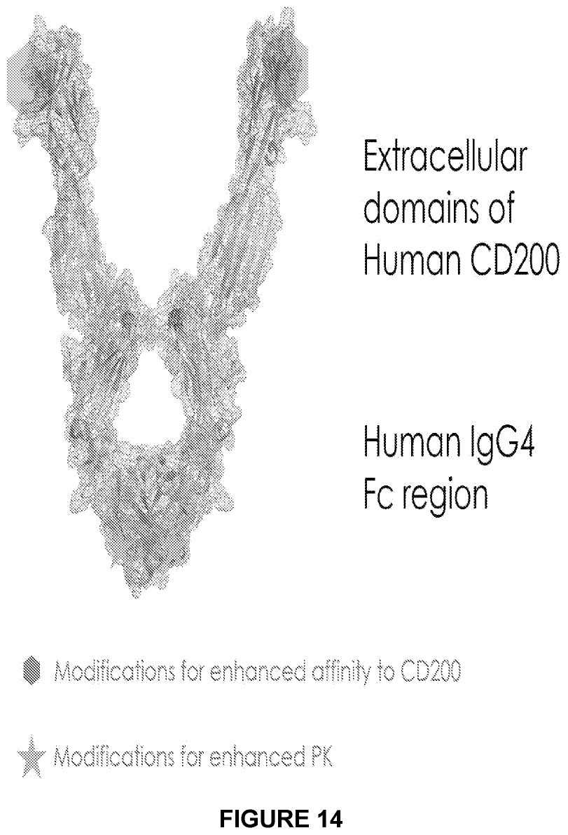

A diagram of the inventive Fc fusion protein, ARQ-234, is shown in FIG. 14.

The binding of ARQ-234 to U937-CD200R cells was compared to a wild type control CD200-Fc (DS-155) at 1, 4 and 24 hrs at human physiological temperature (37° C.) and binding was detected using flow cytometry with an anti-human IgG antibody. As shown in FIG. 4, ARQ-234 binding to U937-CD200R cells demonstrated a more potent target engagement compared to DS-155, a wild type huCD200-Fc construct.

1. A fusion protein comprising:
(i) a mutated CD200 portion comprising mutations at amino acid residue positions 130 and 131, wherein said mutations are K130Y and I131Y; and
(ii) a non-CD200 portion, wherein said non-CD200 portion is an IgG4 Fc fragment and comprises S228P, M428L and N434S mutations according to the EU numbering system and deletion of the first 5 amino acids of the hinge,
wherein Glycine 232 of the mutated CD200 portion is directly fused to the non-CD200 IgG4 Fc fragment at amino acid 6 according to the IMGT numbering system.

2. The fusion protein of claim 1, wherein the fusion protein comprises the amino acid sequence of SEQ ID NO: 1.

3. The fusion protein of claim 1, wherein the fusion protein consists of the amino acid sequence of SEQ ID NO: 1.

4. The fusion protein of claim 1, wherein the mutated CD200 portion comprises an N terminal signal sequence representing the first 30 amino acids of the CD200 portion.

5. The fusion protein of claim 3, wherein the N-terminal signal sequence is a human IgG heavy chain signal peptide.

6. The fusion protein of claim 5, wherein the N-terminal signal sequence comprises the amino acid sequence of SEQ ID NO: 3.

7. The fusion protein of claim 4, wherein the fusion protein comprises the amino acid sequence of SEQ ID NO: 2.

8. The fusion protein of claim 4, wherein the N-terminal signal sequence is cleaved prior to secretion from a cell.

9. The fusion protein of claim 1, which is a modulator of the CD200 receptor.

10. The fusion protein of claim 1, which is an agonist of the CD200 receptor.

11. The fusion protein of claim 1, wherein the fusion protein inhibits cytokine secretion.

12. The fusion protein of claim 11, wherein the cytokine is IL-6.

13. The fusion protein of claim 11, wherein the cytokine is IL-8.

14. The fusion protein of claim 11, wherein the cytokine is TNFα.

15. The fusion protein of claim 1, wherein the fusion protein inhibits ERK activation to a greater extent than wild-type CD200-Fc fusion protein.

16. A polynucleotide encoding the fusion protein of claim 1.

17. A composition comprising the fusion protein of claim 1, and a pharmaceutically acceptable carrier.

18. A method of treating a subject having an autoimmune disease, an allergic disease, a neurodegenerative disorder, neuropathic pain, an inflammatory disorder, Th2-induced airway inflammation, or diabetic neuropathy, comprising administering to the subject the fusion protein of claim 1 or a polynucleotide encoding the fusion protein.

19. The method of claim 18, wherein the subject has hay fever, allergic rhinitis, allergic contact dermatitis, seasonal allergies, anaphylaxis, food allergies, asthma, or atopic dermatitis.

20. The method of claim 19, wherein the subject has an autoimmune disease affecting a neuromuscular system, vascular system, eye, skin, digestive tract, lung, kidney, liver, peripheral or central nervous system, bone, cartilage or joints.

21. The method of claim 20, wherein the subject has acute disseminated encephalomyelitis (ADEM); acute necrotizing haemorrhagic leukoencephalitis; Addison's disease; agammaglobulinemia; alopecia areata; amyloidosis; ankylosing spondylitis; anti-GBM/anti-TBM nephritis; antiphospholipid syndrome (APS); asthma, atopic dermatitis; Autoimmune angioedema; autoimmune aplastic anemia; autoimmune dysautonomia; autoimmune hepatitis; autoimmune hyperlipidemia; autoimmune immunodeficiency; autoimmune inner ear disease (AIED); autoimmune myocarditis; autoimmune oophoritis; autoimmune pancreatitis; autoimmune retinopathy; autoimmune thrombocytopenic purpura (ATP); autoimmune thyroid disease; autoimmune urticarial; axonal & neuronal neuropathies; Balo disease; Behcet's disease; bullous pemphigoid and related autoimmune blistering diseases; cardiomyopathy; Castleman disease; celiac disease (such as refractory celiac disease type II); Chagas disease; chronic idiopathic urticaria; chronic inflammatory demyelinating polyneuropathy (CIDP); chronic recurrent multifocal ostomyelitis (CRMO); chronic spontaneous urticaria; Churg-Strauss syndrome; cicatricial pemphigoid/benign mucosal pemphigoid; Crohn's disease; Cogans syndrome; cold agglutinin disease; congenital heart block; Coxsackie myocarditis; CREST disease; essential mixed cryoglobulinemia; demyelinating neuropathies; dermatitis herpetiformis; dermatomyositis; Devic's disease (neuromyelitis optica); diabetic neuropathy; discoid lupus; Dressler's syndrome; endometriosis; eosinophilic esophagitis; eosinophilic fasciitis; erythema nodosum; experimental allergic encephalomyelitis; Evans syndrome; fibrosing alveolitis; giant cell arteritis (temporal arteritis); giant cell myocarditis; glomerulonephritis; Goodpasture's syndrome; granulomatosis with polyangiitis (GPA) (formerly called Wegener's granulomatosis); graft-versus-host disease (GvHD); Graves' disease; Guillain-Barre syndrome; Hashimoto's encephalitis; Hashimoto's thyroiditis; hemolytic anemia; Henoch-Schonlein purpura; herpes gestationis; hypogammaglobulinemia; Hidradenitis supporativa (HS); idiopathic thrombocytopenia purpura (ITP); IgA nephropathy; IgG4-related sclerosing disease; immunoregulatory lipoproteins; inclusion body myositis; inflammatory bowel disorder (IBD); inflammatory skin disease; interstitial cystitis; juvenile arthritis; juvenile diabetes (type 1 diabetes); juvenile myositis; Kawasaki syndrome; Lambert-Eaton syndrome; leukocytoclastic vasculitis; lichen planus; lichen sclerosus; ligneous conjunctivitis; linear IgA disease (LAD); lupus (SLE); lyme disease, chronic; macrophage activation syndrome (MAS); mastocytosis; Meniere's disease; microscopic polyangiitis; mixed connective tissue disease (MCTD); Mooren's ulcer; Mucha-Habermann disease; multiple sclerosis; myasthenia gravis; myositis; narcolepsy; neuromyelitis optica (Devic's); neutropenia; ocular cicatricial pemphigoid; optic neuritis; palindromic rheumatism; PANDAS (Pediatric Autoimmune Neuropsychiatric Disorders Associated with Streptococcus); Palmoplantar pustulosis (PPP); paraneoplastic cerebellar degeneration; paroxysmal nocturnal hemoglobinuria (PNH); Parry Romberg syndrome; Parsonnage-Turner syndrome; pars planitis (peripheral uveitis); pemphigus; peripheral neuropathy; perivenous encephalomyelitis; pernicious anemia; POEMS syndrome; polyarteritis nodosa; type I, II, & III autoimmune polyglandular syndromes; polymyalgia rheumatic; polymyositis; postmyocardial infarction syndrome; postpericardiotomy syndrome; progesterone dermatitis; primary biliary cirrhosis; primary sclerosing cholangitis; psoriasis; psoriatic arthritis; idiopathic pulmonary fibrosis; pyoderma gangrenosum; pure red cell aplasia; Raynauds phenomenon; reactive arthritis; reflex sympathetic dystrophy; Reiter's syndrome; relapsing polychondritis; restless legs syndrome; retroperitoneal fibrosis; rheumatic fever; rheumatoid arthritis; sarcoidosis; Schmidt syndrome; scleritis; scleroderma; Sjogren's syndrome; sperm & testicular autoimmunity; stiff person syndrome; subacute bacterial endocarditis (SBE); Susac's syndroms; sympathetic ophthalmia; Takayasu's arteritis; temporal arteritis/giant cell arteritis; thrombocytopenia purpura (TTP); Tolosa-Hunt syndrome; transverse myelitis; type 1 diabetes; ulcerative colitis; undifferentiated connective tissue disease (UCTD); uveitis; vasculitis; vesiculobullous dermatosis; or vitiligo.

22. The method of claim 18, wherein the fusion protein or the polynucleotide is administered as the sole therapeutic agent.

23. The method of claim 18, wherein the fusion protein is administered in combination with one of more other pharmaceutical agents indicated for treatment of an autoimmune disease, an allergic disease, a neurodegenerative disorder, neuropathic pain, an inflammatory disorder, Th2-induced airway inflammation, or diabetic neuropathy.

24. The method of claim 18, wherein the fusion protein is administered in combination with one or more immunosuppressive agents or adjuvants in immunosuppression therapy.

25. The method of claim 24, wherein the fusion protein is administered in combination with azathioprine, a methotrexate, a cyclosporine, a monoclonal antibody, a corticosteroid, or a combination thereof.

26. The method of claim 25, wherein the monoclonal antibody is basiliximab, daclizumab, or muromonab.

27. (canceled)


SEQUENCE LISTING (4 sequences)

SEQ ID NO: 1 - protein, 426 aa, Homo sapiens
QVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 2 - protein, 456 aa, Homo sapiens
MERLVIRMPFSHLSTYSLVWVMAAVVLCTAQVQVVTQDEREQLYTPASLKCSLQNAQEALIVTWQKKKAVSPENMVTFSENHGVVIQPAYKDKINITQLGLQNSTITFWNITLEDEGCYMCLFNTFGFGYYSGTACLTVYVQPIVSLHYKFSEDHLNITCSATARPAPMVFWKVPRSGIENSTVTLSHPNGTTSVTSILHIKDPKNQVGKEVICQVLHLGTVTDFKQTVNKGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 3 - protein, 19 aa, Homo sapiens
MEFGLSWLFLVAILKGVQC

SEQ ID NO: 4 - DNA, 1374 bp, Homo sapiens
  source 1..1374: mol_type = other DNA
atggaacggctggtcatcaggatgcccttcagccacctgtctacctacagcctcgtgtgggttatggccgccgtggttctgtgtacagctcaggtgcaggtcgtgacccaggatgagagagagcagctgtatacccctgccagcctgaagtgttccctgcagaatgcccaagaggccctgatcgtgacctggcagaagaagaaggctgtctcccctgagaacatggtcaccttctccgagaatcacggcgtcgtgatccagcctgcctacaaggacaagatcaacatcacacagctgggcctgcagaactccaccatcaccttttggaacatcaccctggaagatgagggctgctacatgtgcctgttcaacaccttcggcttcggctactactctggcaccgcttgtctgaccgtgtacgtgcagcctatcgtgtccctgcactacaagttctccgaggaccacctgaatatcacctgttccgccaccgccagacctgctcctatggtgttttggaaggtgcccagatccggcatcgagaacagcaccgtgacactgtctcaccctaacggcaccacctccgtgacctccatcctgcacatcaaggaccccaagaatcaagtgggcaaagaagtgatctgtcaggttctgcacctgggcacagtgaccgacttcaagcagaccgtgaacaagggccctccttgtcctccatgtcctgctccagaatttctcggcggaccctccgtgttcctgtttcctccaaagcctaaggacaccctgatgatctctcggacccctgaagtgacctgcgtggtggtggatgtgtctcaagaggaccccgaggtgcagttcaattggtacgtggacggcgtggaagtgcacaacgccaagaccaagcctagagaggaacagttcaactccacctacagagtggtgtccgtgctgaccgtgctgcaccaggattggctgaacggcaaagagtacaagtgcaaggtgtccaacaagggactgccctccagcatcgaaaagaccatctccaaggctaagggccagcctcgggaacctcaggtttacaccctgcctccaagccaagaggaaatgaccaagaaccaggtgtccctgacctgcctggtcaagggcttctacccttccgacattgccgtggaatgggagtctaacggccagccagagaacaactacaagaccacacctcctgtgctggactccgacggctccttctttctgtactctcgcctgaccgtggacaagtctaggtggcaagagggcaacgtgttctcctgctctgtgctgcacgaggccctgcactctcactacacccagaagtccctgtctctgtctctgggcaagtgatga